(19) United States
(12) Patent Application Publication
(10) Pub. No.: US 2009/0060920 A1
(43) Pub. Date: Mar. 5, 2009

(54) DESACYL GHRELIN ANTIBODIES AND THERAPEUTIC USES THEREOF

(75) Inventors: Derrick Ryan Witcher, Fishers, IN (US); Kristine Kay Kikly, Fortville, IN (US); Joseph Vincent Manetta, Indianapolis, IN (US)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY

(21) Appl. No.: 11/577,530

(22) PCT Filed: Nov. 8, 2005
(86) PCT No.: PCT/US05/40468

§ 371 Date: Apr. 19, 2007

Related U.S. Application Data
(60) Provisional application No. 60/627,954, filed on Nov. 15, 2004.

(51) Int. Cl. A61K 39/395; C07K 16/26; A61P 3/04; A61P 3/10

(52) U.S. Cl. 424/145.1; 530/388.1

(57) ABSTRACT

A neutralizing epitope is identified within amino acids 1-3 of desacyl ghrelin. Antibodies that bind this epitope fall within the scope of the invention and can be murine, chimeric, or humanized antibodies, immunoconjugates of the antibodies, or antigen-binding fragments thereof. The antibodies of the invention are useful for the treatment or prevention of obesity and related disorders including, for example, Type II non-insulin dependent diabetes mellitus (NIDDM), Prader-Willi syndrome, eating disorders, hyperphagia, and impaired satiety. Additionally, such antibodies can be useful for the treatment or prevention of other disorders, including anxiety, gastric motility disorders (including e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders by administering a therapeutically effective amount of an anti-desacyl ghrelin monoclonal antibody of the invention.

### BACKGROUND OF THE INVENTION

1. Field of the Invention

The present invention is in the field of medicine, particularly in the field of monoclonal antibodies against ghrelin. More specifically the invention relates to neutralizing anti-ghrelin monoclonal antibodies that preferentially bind the desacyl form of ghrelin or precursors thereof but do not bind, or poorly bind, the acylated form of ghrelin. The antibodies of the invention can be murine, chimeric, or humanized antibodies, immunoconjugates of the antibodies, or antigen-binding fragments thereof. The antibodies of the invention are useful in mammals for treating obesity, or for the treatment of conditions wherein the presence of ghrelin, specifically desacyl ghrelin, causes or contributes to undesirable pathological effects, or wherein a decrease in desacyl ghrelin levels, contributes to a desirable therapeutic effect.

2. Description of Related Art

Ghrelin is a 28 amino acid peptide with an n-octanoyl modification at the amino acid at position three (see SEQ ID NO: 17). The ghrelin hormone, when acylated, binds the growth hormone secretagogue receptor (GHS-R1a) in the pituitary, resulting in release of growth hormone. The des-acyl form of ghrelin does not bind the GHS-R1a receptor. (Kojima, et al., Nature 402:656-660, 1999). Ghrelin's other actions include stimulation of prolactin and adrenocorticotropic hormone (ACTH) secretion, effects on the pituitary-gonadal axis, stimulation of appetite, control of energy balance, effects on sleep and behavior, control of gastric motility and acid secretion, effects on exocrine and endocrine pancreatic function and glucose metabolism, effects on the cardiovascular system, and modulation of proliferation of neoplastic cells. (Encyclopedia of Endocrine Diseases (2004), Vol. 3, 295-299. Editor: Martini, Luciano).

The acylated form of ghrelin leads to fat deposition when administered to mice (Tschop, M. et al., Nature 407: 908-913, 2000). Ghrelin is synthesized primarily in the stomach and circulated in the blood. Ghrelin serum levels increase during food deprivation in animals (Kojima, et al., Nature 402:656-660, 1999), peak prior to eating (Cummings, et al., NEJM, 346:1623-1630, 2002), and decrease upon refeeding (Shiiya, et al., J. Clin. Endocrinol. Metab. 87:240-244, 2002). It has been shown that persons who underwent gastric bypass surgery and lost up to 36% of their body weight had greatly reduced ghrelin levels and loss of pre-meal peaks in ghrelin secretion. Persons with Prader-Willi syndrome, a genetic disorder that causes severe obesity with uncontrollable appetite, have extremely high levels of ghrelin. (Cummings, et al., NEJM, 346:1623-1630, 2002). These observations indicate that ghrelin plays a key role in motivating feeding. Additionally, ghrelin is believed to signal the hypothalamus when an increase in metabolic efficiency is required. (Muller, et al., Clin Endocrinol. 55:461-467, 2001).

There are presently limited treatments for obesity. Current treatment options to manage weight include dietary therapy, increased physical activity, and behavior therapy. Unfortunately, these treatments are largely unsuccessful, with a failure rate reaching 95%. This failure can be due to the fact that the condition is strongly associated with genetically inherited factors that contribute to increased appetite, preference for highly caloric foods, reduced physical activity, and increased lipogenic metabolism. This indicates that people inheriting these genetic traits are prone to becoming obese regardless of their efforts to combat the condition. Gastric bypass surgery is available to a limited number of obese persons. However, this type of surgery involves a major operation, can lead to emotional problems, and cannot be modified readily as patient needs demand or change. Additionally, even this attempted remedy can sometimes fail (see, e.g., Kriwanek, Langenbecks Archiv. Fur Chirurgie, 38: 70-74, 1995). Drug therapy options are few and of limited utility. Moreover, chronic use of these drugs can lead to tolerance, as well as side effects from their long term administration. And, when the drug is discontinued, weight often returns.

There is a tremendous therapeutic need for a means to treat obesity, obesity-related disorders, as well as other eating disorders. Due to its role in inducing feeding, ghrelin is a desirable target for therapeutic intervention. In particular, a monoclonal antibody against ghrelin can provide such a therapy. Of particular importance therapeutically is a humanized form of such a monoclonal antibody. Additionally, ghrelin is highly conserved in sequence and in function across species; therefore, not only can such an antibody be useful for the treatment of such disorders in humans, but also in other mammals including, e.g., domestic animals (e.g., canine) and food-source animals (e.g., bovine, porcine and ovine). Such an anti-ghrelin antibody can be useful for the treatment of obesity and related disorders including, for example, Type II non-insulin dependent diabetes mellitus (NIDDM), Prader-Willi syndrome, eating disorders, hyperphagia, and impaired satiety. Additionally, such an antibody can be useful for the treatment or prevention of other disorders, including anxiety, gastric motility disorders (including, e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders. Finally, an anti-ghrelin monoclonal antibody of the invention can be useful for the treatment or prevention of any disease or disorder which benefits from lower levels or lower activity of desacyl ghrelin.

International patent publication number WO 01/07475 (EP1197496) teaches the ghrelin amino acid sequence of various species, including human, and discloses that ghrelin is acylated, typically with O-n-octanoic acid, at the third amino acid from the amino terminus, which is serine in native human ghrelin. WO 01/07475 also indicates that the amino terminal four amino acids of ghrelin are essential for the receptor binding activity of ghrelin. The application further teaches antibodies directed against fatty acid-modified peptides of ghrelin, which peptides induce signal transduction, and the use of such antibodies for assaying or detecting ghrelin.

International patent publication number WO 01/87335 teaches the use of agents that specifically bind ghrelin, including anti-ghrelin antibodies, for the treatment of obesity.

International patent publication number WO 2005/016951, entitled “Anti-Ghrelin Antibodies” and assigned to Eli Lilly and Company, teaches monoclonal anti-ghrelin antibodies that preferentially bind acylated human ghrelin with respect to unacylated human ghrelin, and are useful for treatment of obesity and obesity-related disorders. Such antibodies include murine, chimeric, and humanized antibodies.

International patent publication number WO 03/051389 teaches that administration of desacyl ghrelin can prevent or reduce postprandial induction of insulin resistance by antagonizing some ghrelin actions, and can reduce body weight in some patients.

Murakami et al. (J. Endocrinology 174:283-288, 2002) administered to obese rats by intracerebroventricular injection a polyclonal anti-ghrelin antibody raised against the acylated amino-terminal eleven amino acids of rat ghrelin. The authors were able to demonstrate a subsequent decrease in both food intake and body weight by the rats.

Broglio et al. (Journal of Clinical Endocrinology & Metabolism 89(6):3062-3065, 2004) have found that desacyl ghrelin counteracts the metabolic but not the neuroendocrine effects of acylated ghrelin. Specifically, they found that desacyl ghrelin does not affect the growth hormone, prolactin, and ACTH response to acylated ghrelin, but is able to antagonize the effects of acylated ghrelin on insulin secretion and glucose levels in humans. This indicates that ghrelin could have a dual effect on insulin secretion/sensitivity and glucose homeostasis depending on whether or not it is acylated. Finally, desacyl ghrelin has cardiovascular actions, the ability to modulate cell proliferation, and has a stimulatory effect on adipogenesis that is exerted directly at the adipocyte level.

There are presently limited effective treatments for disorders or conditions that would benefit from a decrease in desacyl ghrelin or a decrease in total ghrelin levels. A monoclonal antibody to desacyl ghrelin can provide a beneficial treatment for such disorders. Of particular therapeutic utility are chimeric or humanized forms of such a monoclonal antibody. Ghrelin is highly conserved in sequence and in function across species. Therefore, not only can such an antibody be useful for the treatment of such disorders in humans, but also in other mammals including, e.g., domestic animals (e.g., canine and feline), sports animals (e.g., equine), and food-source animals (e.g., bovine, porcine, and ovine) particularly when framework and constant regions of the antibody substantially originate from the animal species in which the antibody is to be used therapeutically.

The present invention provides an anti-desacyl ghrelin monoclonal antibody able to preferentially bind to a desacyl ghrelin.

### SUMMARY OF THE INVENTION

Anti-desacyl ghrelin monoclonal antibodies, or antigen-binding fragments thereof, that preferentially bind desacyl ghrelin from a mammalian source are provided by the present invention. Such antibodies are referred to herein as “monoclonal antibodies of the invention” or “antibodies of the invention.” A monoclonal antibody of the invention can be murine, chimeric, or humanized, immunoconjugates of such antibodies, or antigen-binding fragments thereof. Preferably, a monoclonal antibody of the invention exists in a homogeneous or substantially homogeneous population. Preferably, a monoclonal antibody of the invention binds desacyl ghrelin (either the proprotein or the mature form of the protein) within the domain spanning amino acids 1-3 (SEQ ID NO: 17) and thereby antagonizes or neutralizes at least one in vitro, in vivo, or in situ biological activity or property associated with desacyl ghrelin or a portion thereof.

A monoclonal antibody of the present invention preferentially binds desacyl ghrelin over (compared to) acylated ghrelin. Preferably, such antibody binds desacyl ghrelin with greater affinity or specificity than which it binds acylated ghrelin as determined, for example, by ELISA assay, competitive ELISA assay, or K_{D} values in a BIAcore® assay. Furthermore, a monoclonal antibody of the invention can have more favorable Kₒₙ, K_{off}, or Kₐ values with respect to binding desacyl ghrelin than with respect to binding acylated ghrelin. Preferably, an antibody of the invention is non-cross-reactive with acylated ghrelin, or is cross-reactive at a level of about 5%, 4%, 3%, 2%, 1%, or less with acylated ghrelin. Antibodies of the present invention preferably have K_{D} values in a BIAcore® assay of about 1×10⁻⁹ M, about 1×10⁻¹⁰ M, about 1×10⁻¹¹ M, or about 1×10⁻¹² M, i.e., in the range of from about 1×10⁻⁹ M to about 1×10⁻¹² M.

In one embodiment, an anti-desacyl monoclonal antibody, or an antigen-binding fragment thereof, of the present invention preferentially binds desacyl ghrelin or desacyl ghrelin proprotein compared to acylated ghrelin, and either:
a) is cross-reactive with acylated ghrelin at a level of about 5% or less; or
b) binds desacyl ghrelin with an affinity at least about 20-fold greater than it binds acylated ghrelin; or
c) has a dissociation constant, K_{D}, of about 1×10⁻⁹ M; or
d) inhibits a desacyl ghrelin biological activity in vitro or in vivo at less than about 50 μg/ml.

In another embodiment, the monoclonal antibody or antigen-binding fragment thereof of the present invention:
a) is cross-reactive with acylated ghrelin at a level of about 5% or less;
b) binds desacyl ghrelin with an affinity at least about 20-fold greater than it binds acylated ghrelin;
c) has a dissociation constant, K_{D}, of about 1×10⁻⁹ M; and
d) inhibits a desacyl ghrelin biological activity in vitro or in vivo at less than about 50 μg/ml.

In another embodiment, an anti-desacyl monoclonal antibody of the invention comprises a light chain variable region (“LCVR”) polypeptide with an amino acid sequence of SEQ ID NO: 2.

In another embodiment, an anti-desacyl ghrelin monoclonal antibody of the invention comprises a heavy chain variable region (“HCVR”) polypeptide with an amino acid sequence of SEQ ID NO: 10.

In another embodiment, an anti-desacyl ghrelin monoclonal antibody of the invention comprises (a) a LCVR polypeptide with an amino acid sequence of SEQ ID NO: 2 and (b) a HCVR polypeptide with an amino acid sequence of SEQ ID NO: 10.

In another embodiment, a monoclonal antibody of the invention is one that can compete for binding to human desacyl ghrelin or a portion of human desacyl ghrelin with a competing antibody comprising two polypeptides with the sequences shown in SEQ ID NOs: 2 and 10.

In another embodiment, a LCVR of an anti-desacyl ghrelin monoclonal antibody of the invention comprises 1, 2, or 3 peptides selected from the group consisting of peptides with a sequence as shown in SEQ ID NOs: 4, 6 and 8 (see Table 1). Preferably, a peptide with the sequence shown in SEQ ID NO: 4, when present in said antibody, is at LCVR CDR1. Preferably, a peptide with the sequence shown in SEQ ID NO: 6, when present in said antibody, is at LCVR CDR2. Preferably, a peptide with the sequence shown in SEQ ID NO: 8, when present in said antibody, is at LCVR CDR3. The LCVR will further comprise framework sequence. In a humanized antibody for therapeutic use in humans, the framework sequence can be substantially of human origin. In an antibody for use in a non-human animal, the framework region sequence can substantially originate from a sequence in the genome of the animal in which it is to be used therapeutically.

In another embodiment, a HCVR of an anti-desacyl ghrelin monoclonal antibody of the invention comprises 1, 2, or 3 peptides selected from the group consisting of peptides with a sequence as shown in SEQ ID NOS: 12, 14, and 16 (see Table 1). Preferably, a peptide with the sequence shown in SEQ ID NO: 12, when present in said antibody, is at HCVR CDR1. Preferably, a peptide with the sequence shown in SEQ ID NO: 14, when present in said antibody, is at HCVR CDR2. Preferably, a peptide with the sequence shown in SEQ ID NO: 16, when present in said antibody, is at HCVR CDR3. The HCVR will further comprise framework sequence. In a humanized antibody for therapeutic use in humans, the framework sequence can be substantially of human origin. In an antibody for use in a non-human animal, the framework sequence can substantially originate from a sequence in the genome of the animal in which it is to be used therapeutically.

One embodiment of the invention provides an anti-desacyl ghrelin monoclonal antibody comprising the six peptides with the sequences shown in SEQ ID NOs: 4, 6, 8, 12, 14, and 16. Preferably, in said antibody, the peptide with the sequence shown in SEQ ID NO: 4 is located at LCVR CDR1, the peptide with the sequence shown in SEQ ID NO: 6 is located at LCVR CDR2, the peptide with the sequence shown in SEQ ID NO: 8 is located at LCVR CDR3, the peptide with the sequence shown in SEQ ID NO: 12 is located at HCVR CDR1, the peptide with the sequence shown in SEQ ID NO: 14 is located at HCVR CDR2, and the peptide with the sequence shown in SEQ ID NO: 16 is located at HCVR CDR3.

An anti-desacyl monoclonal antibody of the invention can further comprise a heavy chain constant region selected from the group consisting of IgG₁, IgG₂, IgG₃, IgG₄, IgA, IgE, IgM, and IgD. An anti-desacyl ghrelin monoclonal antibody of the invention can further comprise a kappa or lambda light chain constant region. When the antibody is to be used as a human therapeutic, the constant region is preferably, substantially of human origin. When the antibody is to be used as a therapeutic in a non-human animal, the constant region preferably, substantially originates from the animal in which the antibody is to be used as a therapeutic.

An anti-desacyl ghrelin monoclonal antibody of the present invention can comprise, consist essentially of, or consist of an intact antibody (i.e., full length), a substantially intact antibody, a Fab fragment, a F(ab′)₂ fragment, or a single chain Fv fragment.

In a preferred embodiment, an anti-desacyl ghrelin monoclonal antibody of the invention is a chimeric antibody. In a more preferred embodiment, an anti-desacyl ghrelin monoclonal antibody of the invention is a humanized antibody in which framework sequence and constant region sequence present in the antibody are substantially of human origin. The humanized antibody is preferably, a full-length antibody. Alternatively, the framework region, or a portion thereof, and any constant region present in the antibody, can substantially originate from the genome of the animal in which the antibody is to be used as a therapeutic, e.g., domestic animals (e.g., canine, feline), sports animals (e.g., equine), and food-source animals (e.g., bovine, porcine and ovine).

In another embodiment, the invention provides an isolated nucleic acid molecule that comprises a nucleic acid that encodes an LCVR of an antibody of the invention, an HCVR of an antibody of the invention, or an anti-desacyl ghrelin monoclonal antibody of the invention. (Table 5) An exemplary polynucleotide encoding an LCVR of the invention has the sequence shown in SEQ ID NO: 1. An exemplary polynucleotide encoding an HCVR of the invention has the sequence shown in SEQ ID NO: 9.

In another embodiment, the invention provides a vector, preferably, (but not limited to) a plasmid, a recombinant expression vector, a yeast expression vector, or a retroviral expression vector comprising a polynucleotide encoding an anti-desacyl ghrelin monoclonal antibody of the invention. Alternatively, a vector of the invention comprises a polynucleotide encoding an LCVR and/or a polynucleotide encoding an HCVR of the invention. When both an LCVR and a HCVR encoding sequence are present in the same vector, they can be transcribed from one promoter to which they are both operably linked, or they can be transcribed independently, each from a separate promoter to which it is operably linked. If the sequences encoding LCVR and HCVR are present in the same vector and transcribed from one promoter to which they are both operably linked, the LCVR can be 5′ to the HCVR or the LCVR can be 3′ to the HCVR. Furthermore, the LCVR and HCVR coding region in the vector can be separated by a linker sequence of any size or content. Preferably, such linker, when present, is a polynucleotide encoding an internal ribosome entry site.

In another embodiment, the invention provides a host cell comprising a nucleic acid molecule of the present invention. Preferably, a host cell of the invention comprises one or more vectors or constructs comprising a nucleic acid molecule of the present invention. The host cell of the invention is a cell into which a vector of the invention has been introduced (e.g., via transformation, transduction, infection), said vector comprising a polynucleotide encoding an LCVR of an antibody of the invention and/or a polynucleotide encoding a HCVR of the invention. The invention also provides a host cell into which two vectors of the invention have been introduced, one comprising a polynucleotide encoding an LCVR of an antibody of the invention, and one comprising a polynucleotide encoding a HCVR present in an antibody of the invention, each operably linked to a promoter sequence. The host cell types include mammalian, bacterial, plant, and yeast cells. Preferably, the host cell is a CHO cell, a COS cell, an SP2/0 cell, an NS0 cell, a yeast cell, or a derivative or progeny of any preferred cell type.

In another embodiment, the invention provides a method of preparing an anti-desacyl ghrelin monoclonal antibody of the invention, comprising maintaining a host cell of the invention (i.e., a host cell that has been transformed, transduced, or infected with a vector (or vectors) of the invention) under conditions appropriate for expression of a monoclonal antibody of the invention, whereby such antibody is expressed. The method can further comprise the step of isolating the monoclonal antibody of the invention from the cell or preferably, from the culture medium in which such cell is grown.

The present invention encompasses the process of producing an antibody of the invention by injecting a non-human animal, preferably, a rodent, more preferably, a mouse, with (i) an immunogenic peptide consisting of a peptide with a sequence as shown in SEQ ID NO: 17 or (ii) an immunogenic peptide consisting of 27, 26, 25, 24, 23, 22, 21, 20, 19, 18, 17, 16, 15, 14, 13, 12, 11, 10, 9, 8, 7, 6, 5, 4, or 3 contiguous amino acids of a peptide with a sequence as shown in SEQ ID NO: 17. Preferably, such immunogenic peptide spans at least amino acid residues 1-3 of mature desacyl ghrelin, where the third amino acid differs from the amino acid present at the equivalent position of acylated ghrelin in that it is not acylated, or (iii) an immunogenic peptide consisting of amino acids 1-3 of the mature form of desacyl ghrelin of any mammal, or (iv) an immunogenic peptide consisting of amino acids 1-3 of the mature form of desacyl ghrelin of any mammal. Preferably, said immunogenic peptide spans amino acid residues in which the third amino acid differs from the amino acid present at the equivalent position of acylated ghrelin (in that it is not acylated) in the same mammal. Anti-desacyl ghrelin monoclonal antibodies are generated from the immunized animals using any method known in the art, preferably, by hybridoma synthesis. The anti-desacyl ghrelin monoclonal antibodies are screened by any method available in the art (e.g., phage display, ribosome display, yeast display, bacterial display, ELISA assay) for binding to mature desacyl ghrelin, or a portion thereof comprising the immunogenic peptide, or to the immunogenic peptide. Anti-desacyl ghrelin monoclonal antibodies are selected that specifically or preferentially bind desacyl ghrelin compared with their binding to acylated ghrelin. The invention further embodies a monoclonal antibody made by this process. Preferably, such monoclonal antibody binds desacyl ghrelin at least about 5, 10, 20, 30, 40, 50, 60, 70, 80, 90, or about 100-fold greater (about 5- to about 100-fold greater) than it binds acylated ghrelin, more preferably, at least about 150, 200, or 250-fold greater (about 150- to about 250-fold greater, for a total of about 5- to about 250-fold greater) than it binds acylated ghrelin, as determined by a method known to one of skill in the art, e.g., by ELISA, competition ELISA, or K_{D} values in a BIAcore® assay. Most preferably, the monoclonal antibodies of the present invention do not bind acylated ghrelin above background levels in any binding assay available in the art.

It is contemplated that such antibody made by any process of the present invention can be further altered into a chimeric antibody in which at least a portion of the framework and/or constant region originates from a mammal different from that immunized to generate the monoclonal antibody and still fall within the scope of the invention. The antibodies of the invention can be humanized, wherein the murine CDR regions exist within a substantially human framework region, and the constant region, to the extent it is present in the antibody, is also substantially of human origin. The antibodies of the invention can be such that the murine CDR regions exist within a framework region and constant region (to the extent it is present in the antibody) originating from the germline sequence of the animal in which the antibody is to be used therapeutically.

Various forms of the antibodies of the invention are contemplated herein. For example, an anti-desacyl ghrelin monoclonal antibody of the invention can be a full-length antibody (e.g., having an immunoglobulin constant region), or an antibody fragment (e.g., a F(ab′)₂). It is understood that all such forms of the antibodies are encompassed herein within the term “antibody.” Furthermore, the antibody can be labeled with a detectable label, immobilized on a solid phase, and/or conjugated with a heterologous compound (e.g., an enzyme or toxin) according to methods known in the art.

Diagnostic uses for monoclonal antibodies of the present invention are contemplated. In one diagnostic application, the invention provides a method for determining the presence of desacyl ghrelin protein, comprising exposing a test sample suspected of containing the desacyl ghrelin protein to an anti-desacyl ghrelin antibody of the invention and determining specific binding of the antibody to the target in the sample. An anti-desacyl ghrelin antibody of the invention can be used to determine the levels of desacyl ghrelin in test samples by comparing test sample binding values to a standard curve generated by binding said antibody to samples containing known amounts of desacyl ghrelin. The invention further provides a kit, comprising an antibody of the invention and, preferably, instructions for using the antibody to detect desacyl ghrelin protein in, e.g., a test sample.

In another embodiment, the invention provides a pharmaceutical composition, comprising an anti-desacyl ghrelin monoclonal antibody of the invention. The pharmaceutical composition of the invention can further comprise a pharmaceutically acceptable carrier, diluent, or excipient. In such pharmaceutical composition, the anti-desacyl ghrelin monoclonal antibody of the invention is the active ingredient, i.e., it can be the sole active ingredient. Preferably, the pharmaceutical composition comprises a homogeneous or substantially homogeneous population of an anti-desacyl ghrelin monoclonal antibody of the invention. The composition for therapeutic use is sterile, and can be lyophilized.

The invention provides a method of inhibiting desacyl ghrelin activity in a mammal, preferably, a human, in need thereof comprising administering a therapeutically effective amount, or prophylactically effective amount, of an anti-desacyl ghrelin monoclonal antibody or antigen-binding fragment thereof of the invention to said mammal. The invention further provides a method of treating or preventing a disease or disorder ameliorated by the inhibition of signal transduction resulting from the binding of desacyl ghrelin to its receptor, comprising administering to a patient (e.g., a human) in need of such treatment or prevention a therapeutically or prophylactically effective amount of a monoclonal antibody of the invention. As used herein, “treating or preventing” refers to a disease or disorder associated with normal or abnormal desacyl ghrelin levels, or benefited by inhibiting a desacyl ghrelin activity or benefited by a change in the existing desacyl ghrelin level. The invention provides a method for treating disorders associated with prolactin and adrenocorticotropic hormone (ACTH) secretion, effects on the pituitary-gonadal axis, stimulation of appetite, control of energy balance, effects on sleep and behavior, control of gastric motility and acid secretion, effects on exocrine and endocrine pancreatic function and glucose metabolism, and modulation of proliferation of neoplastic cells in a mammal, preferably, a human, in need thereof.

Specifically, diseases or disorders treated or prevented with an antibody of the invention include, but are not limited to, obesity and related disorders including, for example, Type II non-insulin dependent diabetes mellitus (NIDDM), Prader-Willi syndrome, eating disorders, hyperphagia, and impaired satiety. Additionally, such an antibody can be useful for the treatment or prevention of other disorders, including anxiety, gastric motility disorders (including, e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders by administering a therapeutically effective amount of an anti-desacyl ghrelin monoclonal antibody of the invention.

The invention encompasses an anti-desacyl ghrelin monoclonal antibody of the invention for use in the manufacture of a medicament for administration to a mammal, preferably, a human, for the treatment of, e.g., obesity and related disorders including, for example, Type II non-insulin dependent diabetes mellitus (NIDDM), Prader-Willi syndrome, eating disorders, hyperphagia and impaired satiety. Additionally, such an antibody can be useful for the treatment or prevention of other disorders, including anxiety, gastric motility disorders (including e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders in a mammal, preferably, a human, in need thereof by administering to said mammal a therapeutically effective or prophylactically effective amount of an anti-desacyl ghrelin monoclonal antibody of the invention.

The invention also encompasses an article of manufacture, comprising a packaging material and an antibody of the present invention contained within said packaging material, and wherein the packaging material comprises a package insert indicating that the antibody specifically neutralizes a desacyl ghrelin activity, or decreases the level of desacyl ghrelin. Optionally, the package insert further indicates that the antibody preferentially neutralizes a desacyl ghrelin activity with respect to (compared to) acylated ghrelin activity, or preferentially decreases the level of desacyl ghrelin with respect to (compared to) decreasing the level of acylated ghrelin by preferentially binding desacyl ghrelin with respect to (compared to) binding acylated ghrelin.

The present invention encompasses all permutations and combinations of the embodiments disclosed herein.

Further scope of the applicability of the present invention will become apparent from the detailed description provided below. However, it should be understood that the detailed description and specific examples, while indicating preferred embodiments of the present invention, are given by way of illustration only since various changes and modifications within the spirit and scope of the invention will become apparent to those skilled in the art from this detailed description.

### BRIEF DESCRIPTION OF THE DRAWINGS

The above and other aspects, features, and advantages of the present invention will be better understood from the following detailed description taken in conjunction with the accompanying drawings, all of which are given by way of illustration only, and are not limitative of the present invention.

FIG. 1 shows the results of a competition ELISA using acyl ghrelin, desacyl ghrelin, and acyl ghrelin amino acids 2-28 and 3-28, to determine binding of Fab 5611 to desacyl ghrelin, as described in Example 2.

FIG. 2 shows the results of a competition ELISA using desacyl ghrelin, 1-8 (cys) desacyl ghrelin, 4-28 (cys), and 9-28, to determine binding of Fab 5611 to desacyl ghrelin, as described in Example 2.

“2-28 acyl ghrelin” and “3-28 acyl ghrelin” in FIG. 1 refer to acyl ghrelins (SEQ ID NO: 17) missing the first one or two amino acids at the N-terminal end of the molecule, respectively. In FIG. 2, “1-8 (cys) desacyl” refers to a desacyl ghrelin fragment consisting of amino acids 1-8 of SEQ ID NO: 17, with an additional cysteine residue at the C-terminus. “4-28 (cys)” refers to a ghrelin fragment consisting of amino acids 4-28, also with an additional cysteine residue at the C-terminus. “9-28” refers to a ghrelin fragment consisting of amino acids 9-28 of SEQ ID NO: 17.

FIG. 3 shows the results of competition ELISA assays using Fab 5611 and monoclonal antibody E8 with acyl ghrelin and desacyl ghrelin, as described in Example 2.

### DETAILED DESCRIPTION OF THE INVENTION

The following detailed description of the invention is provided to aid those skilled in the in practicing the present invention. Even so, the following detailed description should not be construed to unduly limit the present invention as modifications and variations in the embodiments discussed herein can be made by those of ordinary skill in the art without departing from the spirit or scope of the present inventive discovery.

The contents of each of the references cited herein are herein incorporated by reference in their entirety.

The present invention relates to monoclonal antibodies or functional fragments thereof (e.g., an antigen-binding fragment) that preferentially bind to a mammalian desacyl ghrelin. The antigenic epitope to which monoclonal antibodies of the invention bind is localized to at least amino acid residues 1-3 of mature desacyl ghrelin. In one embodiment, a monoclonal antibody of the invention blocks binding of a ligand (e.g., desacyl ghrelin) to desacyl ghrelin receptor, or inhibits a biological activity of desacyl ghrelin.

An antibody of the present invention preferentially binds mature desacyl ghrelin, or a portion thereof, with an affinity of at least about 1×10⁻⁸ M, preferably, at least about 1×10⁻⁹ M, and more preferably, at least about 1×10⁻¹⁰ M, i.e., in the range from about 1×10⁻⁸ M to about 1×10⁻¹⁰ M. Preferably, the antibodies of the invention do not bind acylated ghrelin greater than background levels of any standard binding assay known in the art. In one embodiment, antibodies of the invention demonstrate inhibition of a desacyl ghrelin biological activity in vitro or in vivo at less than about 150 μg/ml, preferably, less than about 100 μg/ml, more preferably less than about 90, 80, 70, 60, or 50 μg/ml, and even more preferably, less than about 20 μg/ml, i.e., within the range from about 150 μg/ml to about 20 μg/ml, and within any subrange therein.

A full-length antibody as it exists naturally is an immunoglobulin molecule comprised of four peptide chains, two heavy (H) chains (about 50-70 kDa when full length) and two light (L) chains (about 25 kDa when full length), interconnected by disulfide bonds. The amino terminal portion of each chain includes a variable region of about 100-110 or more amino acids primarily responsible for antigen recognition. The carboxy-terminal portion of each chain defines a constant region primarily responsible for effector function.

Light chains are classified as kappa or lambda, and are characterized by a particular constant region. Heavy chains are classified as gamma, mu, alpha, delta, or epsilon, and define the antibody's isotype as IgG, IgM, IgA, IgD, and IgE, respectively. Each heavy chain type is characterized by a particular constant region.

Each heavy chain is comprised of a heavy chain variable region (herein “HCVR”) and a heavy chain constant region. The heavy chain constant region is comprised of three domains (CH₁, CH₂, and CH₃) for IgG, IgD, and IgA; and 4 domains (CH1, CH2, CH3, and CH₄) for IgM and IgE. Each light chain is comprised of a light chain variable region (herein “LCVR”) and a light chain constant region. The light chain constant region is comprised of one domain, CL. The HCVR and LCVR regions can be further subdivided into regions of hypervariability, termed complementarity determining regions (CDRs), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, FR4. The assignment of amino acids to each domain is in accordance with well-known conventions (e.g., Kabat, “Sequences of Proteins of Immunological Interest,” National Institutes of Health, Bethesda, Md. (1991) or Chothia numbering scheme as described in Al-Lazikani et al., J. Mol. Biol. 273:927-948, 1997, see also the internet site http:www.rubic.rdg.ac.uk/˜andrew/bioinf.org/abs). The functional ability of an antibody to bind a particular antigen is determined collectively by the six CDRs. However, even a single variable domain comprising only three CDRs specific for an antigen can have the ability to recognize and bind antigen, although at a lower affinity than a complete Fab.

The term “antibody,” in reference to an anti-desacyl ghrelin monoclonal antibody of the invention (or simply, “monoclonal antibody of the invention”), as used herein, refers to a monoclonal antibody. A “monoclonal antibody” as used herein refers to a rodent, preferably, murine antibody, a chimeric antibody, a primatized antibody, or a humanized antibody. Monoclonal antibodies of the invention can be produced using, e.g., hybridoma techniques well known in the art, as well as recombinant technologies, phage display technologies, synthetic technologies, or combinations of such technologies readily known in the art. The term “monoclonal antibody” as used herein is not limited to antibodies produced through hybridoma technology. “Monoclonal antibody” refers to an antibody that is derived from a single copy or clone, including e.g., any eukaryotic, prokaryotic, or phage clone, and not the method by which it is produced. A “monoclonal antibody” can be an intact (complete or full length) antibody, a substantially intact antibody, or a portion or fragment of an antibody comprising an antigen-binding portion, e.g., a Fab fragment, Fab′ fragment or F(ab′)₂ fragment of a murine antibody, or of a chimeric antibody or of a humanized antibody.

As used herein, the “antigen-binding portion” or “antigen-binding fragment” or “antigen-binding region” or “antigen-binding domain” refers interchangeably herein to that portion of an antibody molecule which contains the amino acid residues that interact with an antigen and confer on the antibody its specificity and affinity for the antigen. This antibody portion includes the “framework” amino acid residues necessary to maintain the proper conformation of the antigen-binding residues. Preferably, the CDRs of the antigen-binding region of the antibodies of the invention will be of murine origin. In other embodiments, the antigen-binding region can be derived from other non-human species including, but not limited to, rabbit, rat, or hamster.

Furthermore, a “monoclonal antibody” as used herein can be a single chain Fv fragment that can be produced by joining the DNA encoding the LCVR and HCVR with a linker sequence. (See, Pluckthun, The Pharmacology of Monoclonal Antibodies, vol. 113, Rosenburg and Moore eds., Springer-Verlag, New York, pp 269-315, 1994). It is understood that regardless of whether fragments are specified, the term “antibody” as used herein includes such fragments as well as single chain forms. As long as the protein retains the ability to specifically or preferentially bind its intended target (i.e., epitope or antigen), it is included within the term “antibody.” Antibodies can or can not be glycosylated and still fall within the bounds of the invention.

A population of “monoclonal antibodies,” refers to a homogeneous or substantially homogeneous (or pure) antibody population (i.e., at least about 90%, 91%, 92%, 93%, 94%, 95%, 96%, more preferably, at least about 97% or 98%, or most preferably, at least 99%, of the antibodies in the population are identical and would compete in an ELISA assay for the same antigen or epitope. Thus, a homogeneous or substantially homogeneous antibody population of the present invention contains about 90% to about 99 or 100% identical antibodies, or any subrange therein.

The term “specifically binds” or “preferentially binds” as used herein refers to the situation in which one member of a specific binding pair does not significantly bind to molecules other than its specific binding partner(s). The term is also applicable where e.g., an antigen-binding domain of an antibody of the invention is specific for a particular epitope that is carried by a number of antigens, in which case the specific antibody carrying the antigen-binding domain will be able to bind to the various antigens carrying the epitope. Accordingly a monoclonal antibody of the invention specifically binds and/or preferentially binds desacyl ghrelin while it does not specifically bind or preferentially bind acylated ghrelin.

In one embodiment, a monoclonal antibody of the invention has less than about 20% cross-reactivity (more preferably, less than about 19, 18, 17, 16, 15, 14, 13, 12, 11, 10, 9, 8, 7, 6, 5, 4, 3, 2 or 1 percent cross-reactivity) with a non-desacyl ghrelin protein or peptide (such as, e.g., acylated ghrelin), i.e., within the range from less than about 20% to less than about 1% cross-reactivity, or any subrange therein, with such a protein or peptide. Preferably, an antibody of the invention binds desacyl ghrelin at least about 5, 10, 20, 30, 40, 50, 60, 70, 80, 90, or 100-fold greater than it binds acylated ghrelin, more preferably at least about 150, 200, or 250-fold greater than it binds acylated ghrelin, as determined e.g., by competition ELISA or BIAcore® assay. Most preferably, the antibodies of the invention do not bind acylated ghrelin at levels greater than background levels of any binding assay available to the art.

The phrases “biological property” or “biological characteristic,” or the terms “activity” or “bioactivity,” in reference to an antibody of the present invention, are used interchangeably herein and include, but are not limited to, epitope/antigen affinity and specificity (e.g., anti-desacyl ghrelin monoclonal antibody binding to desacyl ghrelin or a peptide consisting of the sequence shown in SEQ ID NO: 17), ability to antagonize an activity of desacyl ghrelin in vivo, in vitro, or in situ, the in vivo stability of the antibody, and the immunogenic properties of the antibody. Other identifiable biological properties or characteristics of an antibody recognized in the art include, for example, cross-reactivity, (i.e., with non-human homologs of the targeted peptide, or with other proteins or tissues, generally), and ability to preserve high expression levels of protein in mammalian cells. The aforementioned properties or characteristics can be observed or measured or assessed using art-recognized techniques including, but not limited to, ELISA, competitive ELISA, BIAcore® surface plasmon resonance analysis, in vitro and in vivo neutralization assays without limit, receptor binding, cytokine or growth factor production and/or secretion, Xenopus animal cap development, signal transduction, and immunohistochemistry with tissue sections from different sources including human, primate, or any other source as the need can be.

The term “inhibit” or “neutralize” as used herein with respect to an activity of an antibody of the invention means the ability to substantially antagonize, prohibit, prevent, restrain, slow, disrupt, eliminate, stop, or reverse, e.g., progression or severity of that which is being inhibited including, but not limited to, a biological activity or property, or a disease or a condition.

The term “isolated” when used in relation to a nucleic acid or protein (e.g., an antibody) refers to a nucleic acid sequence or protein that is identified and separated from at least one contaminant with which it is ordinarily associated in its natural source. Preferably, an “isolated antibody” is an antibody that is substantially free of other antibodies having different antigenic specificities (e.g., pharmaceutical compositions of the invention comprise an isolated antibody that specifically binds desacyl ghrelin and is substantially free of antibodies that specifically bind antigens other than desacyl ghrelin).

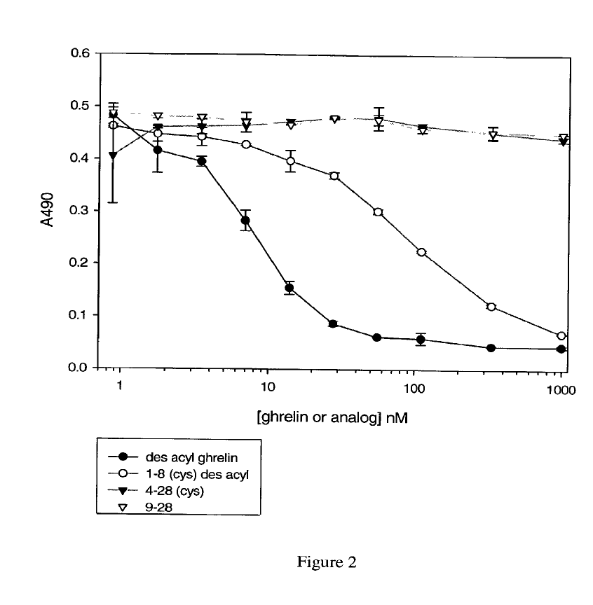

The terms “Kabat numbering” and “Kabat labeling” and “EU index as in Kabat” are used interchangeably herein. These terms, which are recognized in the art, refer to a system of numbering amino acid residues of Ig, e.g., as reflected in FIG. 2 herein (Kabat, et al., Ann. NY Acad. Sci. 190:382-93 (1971); Kabat, et al., Sequences of Proteins of immunological Interest, Fifth Edition, U.S. Department of Health and Human Services, NIH Publication No. 91-3242 (1991)).

A polynucleotide is “operably linked” when it is placed into a functional relationship with another polynucleotide. For example, a promoter or enhancer is operably linked to a coding sequence if it affects the transcription of the sequence.

The terms “individual,” “subject,” and “patient,” used interchangeably herein, refer to a mammal, including, but not limited to, murines, simians, humans, mammalian farm animals, mammalian sport animals, and mammalian pets. Preferably, the term refers to humans.

The term “vector” includes a nucleic acid molecule capable of transporting another nucleic acid to which it has been linked including, but not limited to, plasmids and viral vectors. Certain vectors are capable of autonomous replication in a host cell into which they are introduced while other vectors can be integrated into the genome of a host cell upon introduction into the host cell, and thereby, are replicated along with the host genome. Moreover, certain vectors are capable of directing the expression of genes to which they are operably linked. Such vectors are referred to herein as “recombinant expression vectors” (or simply “expression vectors”), and exemplary vectors are well known in the art.

The term “host cell” includes an individual cell or cell culture that is a recipient of any isolated polynucleotide of the invention or any recombinant vector(s) comprising a HCVR, LCVR, or monoclonal antibody of the invention. Host cells include progeny of a single host cell, and the progeny can not necessarily be completely identical (in morphology or in total DNA complement) to the original parent cell due to natural, accidental, or deliberate mutation and/or change. A host cell includes cells transformed, transduced, or infected in vivo, in situ, or in vitro with a recombinant vector or a polynucleotide expressing a monoclonal antibody of the invention, or a light chain or heavy chain thereof. A host cell that comprises a recombinant vector of the invention (either stably incorporated into the host chromosome or not) can also be referred to as a “recombinant host cell”. Preferred host cells for use in the invention are CHO cells (e.g., ATCC CRL-9096), NS0 cells, SP2/0 cells, COS cells (ATCC e.g., CRL-1650, CRL-1651), and HeLa cells (ATCC CCL-2). Additional host cells for use in the invention include plant cells, yeast cells, other mammalian cells, and prokaryotic cells.

The epitope to which the antibodies of the invention bind (“desacyl ghrelin epitope of the invention”) is localized within the peptide spanning amino acids 1-3 of mature desacyl ghrelin of any mammalian species, preferably, human. Antibodies that bind said epitope specifically or preferentially bind desacyl ghrelin when compared to their binding to acylated ghrelin.

The term “epitope” refers to that portion of a molecule capable of being recognized by and bound by an antibody at one or more of the antibody's antigen-binding regions. Epitopes often consist of a chemically active surface grouping of molecules such as amino acids or sugar side chains, and have specific three-dimensional structural characteristics as well as specific charge characteristics. By “inhibiting epitope” and/or “neutralizing epitope” is intended an epitope, which when in the context of the intact molecule (in this case, desacyl ghrelin) and when bound by an antibody, results in loss or diminution of a biological activity of the molecule or organism containing the molecule, in vivo, in vitro, or in situ.

The term “epitope,” as used herein, further refers to a portion of a polypeptide having antigenic and/or immunogenic activity in an animal, preferably, a mammal, e.g., a mouse or a human. The term “antigenic epitope,” as used herein, is defined as a portion of a polypeptide to which an antibody can specifically bind as determined by any method well known in the art, for example, by conventional immunoassays. Antigenic epitopes need not necessarily be immunogenic, but can be immunogenic. An “immunogenic epitope,” as used herein, is defined as a portion of a polypeptide that elicits an antibody response in an animal, as determined by any method known in the art. (See, e.g., Geysen et al., Proc. Natl. Acad. Sci. USA 81:3998-4002 (1983)). The human desacyl ghrelin antigenic epitope of the present invention has the amino acid sequence shown in SEQ ID NO: 17. A desacyl ghrelin antigenic epitope of the present invention for any mammalian species exists within a peptide consisting of amino acids 1-3 of the mature form of desacyl ghrelin.

The anti-desacyl ghrelin monoclonal antibodies of the invention bind an antigenic epitope discovered to be localized to amino acids 1-3 of mature desacyl ghrelin. A desacyl ghrelin immunogenic and/or antigenic epitope of the invention comprises amino acids 1-3 of the sequence shown in SEQ ID NO: 17. Preferably, the immunogenic epitope spans the third amino acid, which differs from the amino acid present at the equivalent position of acylated ghrelin in that it is not acylated.

An immunogenic epitope of the invention is also contemplated to be an antigenic epitope. The antigenic epitope can possess additional desacyl ghrelin residues outside of amino acids 1-3 of mature desacyl ghrelin, but the monoclonal antibodies of the invention do not require these additional residues to specifically bind desacyl ghrelin. The monoclonal antibodies of the invention bind desacyl ghrelin at least about 5, 10, 20, 30, 40, 50, 60, 70, 80, 90, or 100-fold greater (e.g., greater affinity or greater specificity) than they bind acylated ghrelin, more preferably, at least about 150, 200, or 250-fold greater than they bind acylated ghrelin, as determined e.g., by ELISA assay, competition ELISA assay, or K_{D} values in a Biacore® assay.

The domain spanning amino acids 1-3 (inclusive) of mature desacyl ghrelin or any peptide consisting of an immunogenic epitope as described herein can be used as an immunogenic peptide, preferably, conjugated to a carrier protein, e.g., KLH, to generate monoclonal antibodies of the invention. The immunogenic peptide can be used to immunize a non-human animal, preferably, a mammal, more preferably, a mouse. Then anti-desacyl ghrelin antibodies are isolated from the immunized animal and screened by methods well known in the art to isolate those antibodies that specifically bind amino acids 1-3 of desacyl ghrelin.

Generally, a hybridoma can be produced by fusing a suitable immortal cell line (e.g., a myeloma cell line such as SP2/0) with antibody producing cells of the immunized animal. The antibody producing cell, preferably, those of the spleen or lymph nodes, are obtained from animals immunized with the antigen of interest. The fused cells (hybridomas) can be isolated using selective culture conditions, and cloned by limiting dilution. Cells that produce antibodies with the desired binding properties can be selected by a suitable assay. Methods for such isolation and screening are well known in the art. Selection of antibody fragments from libraries using enrichment technologies such as phage-display (Matthews D J and Wells J A. Science. 260:1113-7, 1993), ribosome display (Hanes, et al., Proc. Natl. Acad. Sci. (USA) 95:14130-5, 1998), bacterial display (Samuelson P., et al., Journal of Biotechnology. 96:129-54, 2002), or yeast display (Kieke M C, et al., Protein Engineering, 10: 1303-10, 1997) has proven to be successful alternatives to classical hybridoma technology (recent reviews: Little M. et al., Immunology Today, 21:364-70, 2000). Antibodies of the invention can be altered to a chimeric or humanized form using methods well known in the art.

Other suitable methods of producing or isolating antibodies that bind amino acids 1-3 of mature desacyl ghrelin, including human or artificial antibodies, can be used, including, for example, methods that select a recombinant antibody (e.g., single chain Fv or Fab) from a library, or which rely upon immunization of transgenic animals (e.g., mice) capable of producing a repertoire of human antibodies (see e.g., Jakobovits et al., Proc. Natl. Acad. Sci. USA, 90:2551-2555, 1993; Jakobovits et al., Nature, 362:255-258, 1993; Lonberg et al., U.S. Pat. No. 5,545,806; Surani et al., U.S. Pat. No. 5,545,807).

Single chain antibodies, and chimeric, humanized, or primatized (CDR-grafted) antibodies, as well as chimeric or CDR-grafted single chain antibodies, and the like, comprising portions derived from different species, are also encompassed by the present invention and the term “antibody.” The various portions of these antibodies can be joined together chemically by conventional techniques, synthetically, or can be prepared as a contiguous protein using genetic engineering techniques. For example, nucleic acids encoding a chimeric or humanized chain can be expressed to produce a contiguous protein. See e.g., U.S. Pat. No. 4,816,567; European Patent No. 0,125,023 B1; U.S. Pat. No. 4,816,397; European Patent No. 0,120,694 B1; WO 86/01533; European Patent No. 0,194,276 B1; U.S. Pat. No. 5,225,539; European Patent No. 0,239,400 B1 and U.S. Pat. Nos. 5,585,089 and 5,698,762. See also, Newman, R. et al. BioTechnology, 10: 1455-1460, 1993, regarding primatized antibody, and Ladner et al., U.S. Pat. No. 4,946,778 and Bird, R. E. et al., Science, 242:423-426, 1988, regarding single chain antibodies.

In addition, functional fragments of antibodies, including fragments of chimeric, humanized, primatized, or single chain antibodies, can also be produced. Functional fragments of the foregoing antibodies retain at least one binding function and/or biological function of the full-length antibody from which they are derived. Preferred functional fragments retain an antigen-binding function of a corresponding full-length antibody (e.g., the ability to bind a mammalian mature form of desacyl ghrelin). Particularly preferred functional fragments retain the ability to inhibit one or more functions or bioactivities characteristic of a mammalian mature desacyl ghrelin, such as a binding activity, a signaling activity, and/or stimulation of a cellular response. For example, in one embodiment, a functional fragment can inhibit the interaction of mature desacyl ghrelin with one or more of its ligands and/or can inhibit one or more receptor-mediated functions.

Antibody fragments capable of binding to a mammalian mature desacyl ghrelin or portion thereof, include, but are not limited to, Fv, Fab, Fab′, and F(ab′)₂ fragments, and are encompassed by the invention. Such fragments can be produced by enzymatic cleavage or by recombinant techniques. For instance, papain or pepsin cleavage can generate Fab or F(ab′)₂ fragments, respectively. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

In a preferred embodiment, the present invention provides an anti-desacyl ghrelin monoclonal antibody resulting from the process described that preferably, binds mature desacyl ghrelin, or a portion thereof, with an affinity of at least about 1×10⁻¹⁰ M, preferably at least about 1×10⁻⁹ M, and more preferably at least about 1×10⁻⁸ M, i.e., in the range from at least about 1×10⁻¹⁰ M to at least about 1×10⁻⁸ M (as determined, e.g., by solid phase BIAcore® surface plasmon resonance assay), and that has the capacity to antagonize a biological activity of a mature desacyl ghrelin.

A preferred monoclonal antibody of the invention has an LCVR comprising a peptide with a sequence of SEQ ID NO: 2 and/or a HCVR comprising a peptide with a sequence of SEQ ID NO: 10. Furthermore, a monoclonal antibody of the invention is one that is competitively inhibited from binding mature human desacyl ghrelin (or a portion thereof) by a monoclonal antibody comprising two polypeptides with the sequences shown in SEQ ID NOS: 2 and 10.

In another embodiment, an LCVR of an anti-desacyl ghrelin monoclonal antibody of the invention comprises 1, 2, or 3 peptides selected from the group consisting of peptides with sequences shown in SEQ ID NOs: 4, 6, and 8 (see Table 1). A HCVR of an anti-desacyl ghrelin monoclonal antibody of the invention comprises 1, 2, or 3 peptides selected from the group consisting of peptides with sequences shown in SEQ ID NOs: 12, 14, and 16.

In a preferred embodiment, an anti-desacyl ghrelin monoclonal antibody of the invention is a chimeric antibody or a humanized antibody. Alternatively, the framework and any constant region present in the antibody can substantially originate from the genome of the animal in which the antibody is to be used as a therapeutic. A preferred antibody is a full-length antibody.

The present invention is also directed to cell lines that express an anti-desacyl ghrelin monoclonal antibody of the invention, or a portion thereof. Creation and isolation of cell lines producing a monoclonal antibody of the invention can be accomplished using standard techniques known in the art. Preferred cell lines include COS, CHO, SP2/0, NS0, and yeast (available from public repositories such as ATCC, American Type Culture Collection, Manassas, Va.).

A wide variety of host expression systems can be used to express an antibody of the present invention, including prokaryotic (bacterial) and eukaryotic expression systems (such as yeast, baculovirus, plant, mammalian, and other animal cells, transgenic animals, and hybridoma cells), as well as phage display expression systems. An example of a suitable bacterial expression vector is pUC119, and a suitable eukaryotic expression vector is a modified pcDNA3.1 vector with a weakened DHFR selection system. Other antibody expression systems are also known in the art and are contemplated herein.

An antibody of the invention can be prepared by recombinant expression of immunoglobulin light and heavy chain genes in a host cell. To express an antibody recombinantly, a host cell is transformed, transduced, infected, or the like with one or more recombinant expression vectors carrying DNA fragments encoding the immunoglobulin light and/or heavy chains of the antibody such that the light and/or heavy chains are expressed in the host cell. The heavy chain and the light chain can be expressed independently from different promoters to which they are operably linked in one vector or, alternatively, the heavy chain and the light chain can be expressed independently from different promoters to which they are operably linked in two vectors, one expressing the heavy chain and one expressing the light chain. Optionally, the heavy chain and light chain can be expressed in different host cells. Preferably, the recombinant antibodies are secreted into the medium in which the host cells are cultured, from which the antibodies can be recovered or purified. Standard recombinant DNA methodologies are used to obtain antibody heavy and light chain genes, incorporate these genes into recombinant expression vectors, and introduce the vectors into host cells. Such standard recombinant DNA technologies are described, for example, in Sambrook, Fritsch, and Maniatis (Eds.), Molecular Cloning; A Laboratory Manual, Second Edition, Cold Spring Harbor, N.Y., 1989; Ausubel, et al (Eds.) Current Protocols in Molecular Biology, Greene Publishing Associates, 1989.

An isolated DNA encoding a HCVR region can be converted to a full-length heavy chain gene by operably linking the HCVR-encoding DNA to another DNA molecule encoding heavy chain constant regions (CH₁, CH₂, and CH₃). The sequences of human heavy chain constant region genes are known in the art. See, e.g., Kabat, et al., Sequences of Proteins of Immunological Interest, Fifth Edition, U.S. Department of Health and Human Services, NIH Publication No. 91-3242 (1991). DNA fragments encompassing these regions can be obtained e.g., by standard PCR amplification. The heavy chain constant region can be of any type, (e.g., IgG, IgA, IgE, IgM, or IgD), class (e.g., IgG₁, IgG₂, IgG₃, and IgG₄) or subclass constant region, and any allotypic variant thereof as described in Kabat (supra). Alternatively, the antigen binding portion can be a Fab fragment, Fab′ fragment, F(ab′)₂ fragment, Fv, or a single chain Fv fragment (scFv). For a Fab fragment heavy chain gene, the HCVR-encoding DNA can be operably linked to another DNA molecule encoding only a heavy chain CH₁ constant region.

An isolated DNA encoding an LCVR region can be converted to a full-length light chain gene (as well as a Fab light chain gene) by operably linking the LCVR-encoding DNA to another DNA molecule encoding a light chain constant region, CL. The sequences of human light chain constant region genes are known in the art. See, e.g., Kabat, supra. DNA fragments encompassing these regions can be obtained by standard PCR amplification. The light chain constant region can be a kappa or lambda constant region.

To create an scFv gene, the HCVR— and LCVR-encoding DNA fragments are operably linked to another fragment encoding a flexible linker, e.g., encoding the amino acid sequence (Gly₄-Ser)₃, such that the HCVR and LCVR sequences can be expressed as a contiguous single-chain protein, with the LCVR and HCVR regions joined by the flexible linker. See, e.g., Bird, et al., Science 242:423-6, 1988; Huston, et al., Proc. Natl. Acad. Sci. USA 85:5879-83, 1988; McCafferty, et al., Nature 348:552-4, 1990.

To express an antibody of the invention, a DNA encoding a partial or full-length light and/or heavy chain, obtained as described above, are inserted into an expression vector such that the gene is operably linked to transcriptional and translational control sequences. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods. Additionally, the recombinant expression vector can encode a signal peptide that facilitates secretion of the anti-desacyl ghrelin monoclonal antibody light and/or heavy chain from a host cell. The anti-desacyl ghrelin monoclonal antibody light and/or heavy chain gene can be cloned into the vector such that the signal peptide is operably linked in-frame to the amino terminus of the antibody chain gene. The signal peptide can be an immunoglobulin signal peptide or a heterologous signal peptide.

In addition to the antibody heavy and/or light chain gene(s), a recombinant expression vector of the invention carries regulatory sequences that control the expression of the antibody chain gene(s) in a host cell. The term “regulatory sequence” is intended to include promoters, enhancers, and other expression control elements (e.g., polyadenylation signals), as needed, that control the transcription or translation of the antibody chain gene(s). The design of the expression vector, including the selection of regulatory sequences, may depend on such factors as the choice of the host cell to be transformed and the level of protein expression desired. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)), and polyoma virus.

In addition to the antibody heavy and/or light chain genes and regulatory sequences, the recombinant expression vectors of the invention can carry additional sequences, such as sequences that regulate replication of the vector in host cells (e.g., origins of replication) and one or more selectable marker genes. The selectable marker gene facilitates selection of host cells into which the vector has been introduced. For example, typically the selectable marker gene confers resistance to drugs, such as G418, hygromycin, or methotrexate, on a host cell into which the vector has been introduced. Preferred selectable marker genes include the dihydrofolate reductase (DHFR) gene (for use in DHFR-minus host cells with methotrexate selection/amplification), the neo gene (for G418 selection), and glutamine synthetase (GS) in a GS-negative cell line (such as NS0) for selection/amplification.

For expression of the light and/or heavy chains, the expression vector(s) encoding the heavy and/or light chains is introduced into a host cell by standard techniques, e.g., electroporation, calcium phosphate precipitation, DEAE-dextran transfection, transduction, infection, and the like. Although it is theoretically possible to express the antibodies of the invention in either prokaryotic or eukaryotic host cells, eukaryotic cells are preferred, most preferably mammalian host cells, because such cells are more likely to assemble and secrete a properly folded and immunologically active antibody. Preferred mammalian host cells for expressing the recombinant antibodies of the invention include Chinese Hamster Ovary (CHO cells) (including DHFR-CHO cells, described in Urlaub and Chasin, Proc. Natl. Acad. Sci. USA 77:4216-20, 1980), used with a DHFR selectable marker, e.g., as described in Kaufman and Sharp, J. Mol. Biol. 159:601-21, 1982, NS0 myeloma cells, COS cells, and SP2/0 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the host cell and/or the culture medium using standard purification methods.

Host cells can also be used to produce portions, or fragments, of intact antibodies, e.g., Fab fragments or scFv molecules, by conventional techniques. It will be understood that variations on the above procedures are within the scope of the present invention. For example, it can be desirable to transfect a host cell with DNA encoding either the light chain or the heavy chain of an antibody of this invention. Recombinant DNA technology can also be used to remove some or all the DNA encoding either or both of the light and heavy chains that is not necessary for binding to desacyl ghrelin. The molecules expressed from such truncated DNA molecules are also encompassed by the antibodies of the invention.

In a preferred system for recombinant expression of an antibody of the invention, a recombinant expression vector encoding both the antibody heavy chain and the antibody light chain is introduced into DHFR—CHO cells by, e.g., calcium phosphate-mediated transfection. Within the recombinant expression vector, the antibody heavy and light chain genes are each operably linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Antibodies, or antigen-binding portions thereof, of the invention can also be expressed in an animal (e.g., a mouse) that is transgenic for human immunoglobulin genes (see, e.g., Taylor, et al., Nucleic Acids Res. 20:6287-95, 1992).

Once expressed, the intact antibodies, their dimers, individual light and heavy chains, or other immunoglobulin forms of the present invention can be purified according to standard procedures in the art, including ammonium sulfate precipitation, ion exchange, affinity, reverse phase, hydrophobic interaction column chromatography, gel electrophoresis, and the like. Substantially pure immunoglobulins of at least about 90%, 92%, 94%, or 96% homogeneity are preferred, with 98% to 99% or greater homogeneity being most preferred, for pharmaceutical uses. Once purified, partially or to homogeneity as desired, the peptides can then be used therapeutically or prophylactically, as directed herein.

As used herein, the term “chimeric antibody” includes monovalent, divalent or polyvalent immunoglobulins. A monovalent chimeric antibody is a dimer formed by a chimeric heavy chain associated through disulfide bridges with a chimeric light chain. A divalent chimeric antibody is a tetramer formed by two heavy chain-light chain dimers associated through at least one disulfide bridge.

A chimeric heavy chain of an antibody for use in humans comprises an antigen-binding region derived from the heavy chain of a non-human antibody specific for desacyl ghrelin, linked to at least a portion of a human heavy chain constant region, such as CH1 or CH2. A chimeric light chain of an antibody for use in humans comprises an antigen binding region derived from the light chain of a non-human antibody specific for desacyl ghrelin, linked to at least a portion of a human light chain constant region (CL).

Antibodies, fragments, or derivatives having chimeric heavy chains and light chains of the same or different variable region binding specificity can also be prepared by appropriate association of the individual polypeptide chains, according to known method steps.

With this approach, hosts expressing chimeric heavy chains are separately cultured from hosts expressing chimeric light chains, and the immunoglobulin chains are separately recovered and then associated. Alternatively, the hosts can be co-cultured and the chains allowed to associate spontaneously in the culture medium, followed by recovery of the assembled immunoglobulin or fragment.

Methods for producing chimeric antibodies are known in the art (see, e.g., U.S. Pat. Nos. 6,284,471; 5,807,715; 4,816,567; and 4,816,397).

In a preferred embodiment, a gene is created which comprises a first DNA segment that encodes at least the antigen-binding region of non-human origin such as functionally rearranged variable (V) region with joining (J) segment, linked to a second DNA segment encoding at least a part of a human constant (C) region as described in U.S. Pat. No. 6,284,471.

Preferably, an antibody of the invention to be used for therapeutic purposes would have the sequence of the framework and constant region as exists in the antibody derived from the mammal in which it would be used as a therapeutic so as to decrease the possibility that the mammal would elicit an immune response against the therapeutic antibody.

Humanized antibodies are of particular interest, since they are considered to be valuable for therapeutic application, avoiding the human anti-mouse antibody response frequently observed with rodent antibodies. The term “humanized antibody” as used herein refers to an immunoglobulin comprising portions of antibodies of different origin, wherein at least one portion is of human origin. For example, the humanized antibody can comprise portions derived from an antibody of nonhuman origin with the requisite specificity, such as a mouse, and from an antibody of human origin, joined together chemically by conventional techniques (e.g., synthetic), or prepared as a contiguous polypeptide using genetic engineering techniques. Preferably, a “humanized antibody” has CDRs that originate from a non-human antibody (preferably, a mouse monoclonal antibody), while framework and constant region, to the extent it is present (or a significant or substantial portion thereof, i.e., at least about 90%, 92%, 94%, 96%, 98%, or 99%) are encoded by nucleic acid sequence information that occurs in the human germline immunoglobulin region (see, e.g., the International ImMunoGeneTics Database) or in recombined or mutated forms thereof whether or not said antibodies are produced in a human cell. A humanized antibody can be an intact antibody, a substantially intact antibody, a portion of an antibody comprising an antigen-binding site, or a portion of an antibody comprising a Fab fragment, Fab′ fragment, F(ab′)₂, or a single chain Fv fragment. It is contemplated that in the process of creating a humanized antibody, the amino acid at either termini of a CDR can be substituted with an amino acid that occurs in the human germline for that segment of adjoining framework sequence.

Humanized antibodies can be subjected to in vitro mutagenesis using methods of routine use in the art (or, when an animal transgenic for human Ig sequences is used, in vivo somatic mutagenesis) and, thus, the framework region amino acid sequences of the HCVR and LCVR regions of the humanized recombinant antibodies are sequences that, while derived from those related to human germline HCVR and LCVR sequences, can not naturally exist within the human antibody germline repertoire in vivo. It is contemplated that such amino acid sequences of the HCVR and LCVR framework regions of the humanized recombinant antibodies are at least about 90%, 92%, 94%, 96%, 98%, or most preferably, at least 99%, identical to a human germline sequence.

Humanized antibodies have at least three potential advantages over non-human and chimeric antibodies for use in human therapy: (i) as the effector portion is human, it can interact better with the other parts of the human immune system (e.g., destroy the target cells more efficiently by complement-dependent cytotoxicity or antibody-dependent cellular cytotoxicity); (ii) the human immune system should not recognize the framework or constant region of the humanized antibody as foreign, and therefore the antibody response against such an injected antibody should be less than that against a totally foreign non-human antibody or a partially foreign chimeric antibody; and (iii) injected non-human antibodies have been reported to have a half-life in the human circulation much shorter than the half-life of human antibodies. Injected humanized antibodies can have a half-life much like that of naturally occurring human antibodies, thereby allowing smaller and less frequent doses to be given.

Humanization can in some instances adversely affect antigen binding of the antibody. Preferably, a humanized anti-desacyl ghrelin monoclonal antibody of the present invention will possess a binding affinity for desacyl ghrelin of not less than about 50%, more preferably not less than about 30%, and most preferably not less than about 25%, 20%, 15%, 10%, or 5% of the binding affinity of the parent murine antibody, i.e., in the range from not less than about 50% to not less than about 5%, preferably Fab 5611, for desacyl ghrelin. Preferably, a humanized antibody of the present invention will bind the same epitope as does Fab 5611 described herein. Such antibody can be identified based on its ability to compete with Fab 5611 for binding to mature desacyl ghrelin or a peptide with the sequence shown in SEQ ID NO: 17.

In general, the humanized antibodies are produced by obtaining nucleic acid sequences encoding the HCVR and LCVR of an antibody that binds a desacyl ghrelin epitope of the invention, identifying the CDRs in said HCVR and LCVR (nonhuman), and grafting such CDR-encoding nucleic acid sequences onto selected human framework-encoding nucleic acid sequences. Preferably, the human framework amino acid sequences are selected such that the resulting antibody is likely to be suitable for in vivo administration in humans. This can be determined, e.g., based on previous usage of antibodies containing such human framework sequence. Preferably, the human framework sequence will not itself be significantly immunogenic.

Alternatively, the amino acid sequences of the frameworks for the antibody to be humanized will be compared to those of known human framework sequences the human framework sequences to be used for CDR-grafting will be selected based on their comprising sequences highly similar to those of the parent antibody, e.g., a murine antibody that binds desacyl ghrelin. Numerous human framework sequences have been isolated and their sequences reported in the art. This enhances the likelihood that the resultant CDR-grafted humanized antibody, which contains CDRs of the parent (e.g., murine) antibody grafted onto selected human frameworks (and possibly also the human constant region) will substantially retain the antigen binding structure and thus retain the binding affinity of the parent antibody. To retain a significant degree of antigen binding affinity, the selected human framework regions will preferably, be those that are expected to be suitable for in vivo administration, i.e., are not immunogenic.

In either method, the DNA sequences encoding the HCVR and LCVR regions of the preferably, murine anti-desacyl ghrelin antibody are obtained. Methods for cloning nucleic acid sequences encoding immunoglobulins are well known in the art. Such methods can, for example, involve the amplification of the immunoglobulin-encoding sequences to be cloned using appropriate primers by polymerase chain reaction (PCR). Primers suitable for amplifying immunoglobulin nucleic acid sequences, and specifically murine HCVR and LCVR sequences have been reported in the literature. After such immunoglobulin-encoding sequences have been cloned, they will be sequenced by methods well known in the art.

Once the DNA sequences encoding the CDRs and frameworks of the antibody that are to be humanized have been identified, the amino acid sequences encoding the CDRs are then identified (deduced based on the nucleic acid sequences and the genetic code and by comparison to previous antibody sequences) and the CDR-encoding nucleic acid sequences are grafted onto selected human framework-encoding sequences. This can be accomplished by use of appropriate primers and linkers. Methods for selecting suitable primers and linkers to prime for ligation of desired nucleic acid sequences are well within the ability of one of ordinary skill in the art.

After the CDR-encoding sequences are grafted onto the selected human framework encoding sequences, the resultant DNA sequences encoding the “humanized” variable heavy and variable light sequences are then expressed to produce a humanized Fv or humanized antibody that binds desacyl ghrelin. Typically, the humanized HCVR and LCVR are expressed as part of a whole anti-desacyl ghrelin antibody molecule, i.e., as a fusion protein with human constant domain sequences whose encoding DNA sequences have been obtained from a commercially available library, or that have been obtained using, e.g., one of the above-described methods for obtaining DNA sequences, or are in the art. However, the HCVR and LCVR sequences can also be expressed in the absence of constant sequences to produce a humanized anti-desacyl ghrelin Fv. Nevertheless, fusion of human constant sequences is potentially desirable because the resultant humanized anti-desacyl ghrelin antibody can possess human effector functions.

Methods for synthesizing DNA encoding a protein of known sequence are well known in the art. Using such methods, DNA sequences that encode the subject humanized HCVR and LCVR sequences (with or without constant regions) are synthesized, and then expressed in a vector system suitable for expression of recombinant antibodies. This can be effected in any vector system that provides for the subject humanized HCVR and LCVR sequences to be expressed as a fusion protein with human constant domain sequences and to associate to produce functional (antigen binding) antibodies or antibody fragments.

Human constant domain sequences are well known in the art, and have been reported in the literature. Preferred human constant light chain sequences include the kappa and lambda constant light chain sequences. Preferred human constant heavy chain sequences include human gamma 1, human gamma 2, human gamma 3, human gamma r, and mutated versions thereof that provide for altered effect or function, e.g., enhanced in vivo half-life, reduced Fc receptor binding, and the like.

If present, human framework regions are preferably, derived from a human antibody variable region having sequence similarity to the analogous or equivalent region of the antigen binding region donor. Other sources of framework regions for portions of human origin of a humanized antibody include human variable consensus sequences (see e.g., Kettleborough, C. A. et al. Protein Engineering 4:773-783 (1991); Carter et al., WO 94/04679. For example, the sequence of the antibody or variable region used to obtain the nonhuman portion can be compared to human sequences as described in Kabat et al. Sequences of Proteins of Immunological Interest, Fifth Edition, NIH, U.S. Government Printing Office (1991). In a particularly preferred embodiment, the framework regions of a humanized antibody chain are derived from a human variable region having at least about 60% overall sequence identity, preferably at least about 70% overall sequence identity, and more preferably at least about 85% overall sequence identity, with the variable region of the nonhuman donor. A human portion can also be derived from a human antibody having at least about 65% sequence identity, and preferably, at least about 70% sequence identity, within the particular portion (e.g., FR) being used, when compared to the equivalent portion (e.g., FR) of the nonhuman donor.

In some instances, humanized antibodies produced by grafting CDRs (from an antibody that binds desacyl ghrelin) onto selected human frameworks can provide humanized antibodies having the desired affinity to desacyl ghrelin. However, it can be necessary or desirable to further modify specific residues of the selected human framework in order to enhance antigen binding. Preferably, those framework residues of the parent (e.g., murine) antibody that maintain or affect combining-site structures will be retained. These residues can be identified by X-ray crystallography of the parent antibody or Fab fragment, thereby identifying the three-dimensional structure of the antigen-binding site.

References further describing methods involved in humanizing a mouse antibody that can be used are, e.g., Queen et al., Proc. Natl. Acad. Sci. USA 88:2869, 1991; U.S. Pat. No. 5,693,761; U.S. Pat. No. 4,816,397; U.S. Pat. No. 5,225,539; and computer programs ABMOD and ENCAD as described in Levitt, M., J. Mol. Biol. 168:595-620, 1983.

Antibodies of the present invention are useful in therapeutic, diagnostic, and research applications as described herein. An antibody of the invention can be used to diagnose a disorder or disease associated with the expression (over-, under- or normal expression) of human desacyl ghrelin. In a similar manner, the antibody of the invention can be used in an assay to monitor desacyl ghrelin levels in a subject being treated for a desacyl ghrelin-associated condition. Diagnostic assays include methods that utilize the antibody of the invention and a label to detect desacyl ghrelin in a sample, e.g., in a human body fluid or in a cell or tissue extract. Binding compositions, such as, e.g., antibodies, are used with or without modification, and are labeled by covalent or non-covalent attachment of a detectable moiety. The detectable moiety can be any one that is capable of producing, either directly or indirectly, a detectable signal. For example, the detectable moiety can be a radioisotope such as, e.g., ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase, or horseradish peroxidase. Any method known in the art for separately conjugating the antibody to the detectable moiety can be employed, including those methods described by Hunter, et al., Nature 144:945, 1962; David, et al., Biochemistry 13: 1014, 1974; Pain, et al., J. Immunol. Meth. 40: 219, 19811; and Nygren, J. Histochem. And Cytochem. 30: 407, 1982.

A variety of conventional protocols for measuring desacyl ghrelin, including e.g., ELISAs, RIAs, and FACS, are known in the art and provide a basis for diagnosing altered or abnormal levels of desacyl ghrelin expression. Normal or standard expression values are established using any art known technique, e.g., by combining a sample comprising a desacyl ghrelin polypeptide with, e.g., antibodies under conditions suitable to form a antigen:antibody complex. The antibody is directly or indirectly labeled with a detectable substance to facilitate detection of the bound or unbound antibody. Suitable detectable substances include various enzymes, prosthetic groups, fluorescent materials, luminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; an example of a luminescent material includes luminol; and examples of a radioactive material include ¹²⁵I, ¹³¹I, ³⁵S, or ³H. (See, e.g., Zola, Monoclonal Antibodies: A Manual of Techniques, CRC Press, Inc. (1987)).

The amount of a standard complex formed is quantitated by various methods, such as, e.g., photometric means. Amounts of desacyl ghrelin polypeptide expressed in subject, control, and samples (e.g., from biopsied tissue) are then compared with the standard values. Deviation between standard and subject values establishes parameters for correlating a particular disorder, state, condition, syndrome, or disease with a certain level of expression (or lack thereof) for a desacyl ghrelin polypeptide.

Once the presence of a disorder, state, condition, syndrome, or disease is established and a treatment protocol is initiated, assays are repeated on a regular basis to monitor the level of desacyl ghrelin expression. The results obtained from successive assays are used to show the efficacy of treatment over a period ranging from several days to months or years. With respect to a particular disorder, the presence of an altered amount of desacyl ghrelin in biopsied tissue or fluid (e.g., serum or urine) from a subject can indicate a predisposition for the development of a disorder, state, condition, syndrome, or disease, or it can provide a means for detecting such a disorder, state, condition, syndrome, or disease prior to the appearance of actual clinical symptoms, or it can define a population more likely to respond therapeutically to an antibody of the invention. A more definitive initial detection can allow earlier treatment, thereby preventing and/or ameliorating further progression of cell proliferation or disease.

An antibody of the invention can be incorporated into pharmaceutical compositions suitable for administration to a subject. Such antibody can be the sole active pharmaceutically active ingredient in such a composition, i.e., antibodies of the present invention can be used alone. Alternatively, antibodies of the present invention can also be used in combinations with one another. Furthermore, the antibody compounds of the present invention can be administered alone or in combination with a pharmaceutically acceptable carrier, diluent, and/or excipients, in single or multiple doses. The pharmaceutical compositions for administration are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents, and the like are used as appropriate. Such compositions are designed in accordance with conventional techniques as described in, e.g., Remington, The Science and Practice of Pharmacy, 19^{th} Edition, Gennaro, Ed., Mack Publishing Co., Easton, Pa., 1995, which provides a compendium of formulation techniques as are generally known to practitioners.

A pharmaceutical composition comprising an anti-desacyl ghrelin monoclonal antibody of the present invention can be administered to a subject at risk for, or exhibiting, pathologies as described herein using standard enteral and parenteral administration techniques including oral, intravenous, intraperitoneal, subcutaneous, pulmonary, transdermal, intramuscular, intranasal, buccal, sublingual, or suppository administration.

A pharmaceutical composition of the invention is preferably a “therapeutically effective amount” or a “prophylactically effective amount” of an antibody, or combination of antibodies, of the present invention. A “therapeutically effective amount” refers to an amount that is effective, at dosages and for periods of time necessary, to achieve the desired therapeutic result. A therapeutically effective amount of the antibody can vary according to factors such as the disease state, age, sex, and weight of the individual, and the ability of the antibody or antibody portion to elicit a desired response in the individual. A therapeutically effective amount is also one in which any toxic or detrimental effect of the antibody are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A therapeutically effective amount is at least the minimal dose, but less than a toxic dose, of an active agent necessary to impart therapeutic benefit to a subject. Stated another way, a therapeutically effective amount is an amount which in mammals, preferably, humans, treats conditions wherein the presence of desacyl ghrelin causes or contributes to undesirable pathological effects, or wherein a decrease in desacyl ghrelin levels results in a beneficial therapeutic effect in a mammal, preferably, a human, including, but not limited to, obesity and related disorders including, for example, Type II non-insulin dependent diabetes mellitus (NIDDM), Prader-Willi syndrome, eating disorders, hyperphagia, and impaired satiety. Additionally, such an antibody can be useful for the treatment or prevention of other disorders, including anxiety, gastric motility disorders (including e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders.

The route of administration of an antibody of the present invention can be oral, parenteral, by inhalation, or topical. Preferably, the antibodies of the invention can be incorporated into a pharmaceutical composition suitable for parenteral administration. The term parenteral as used herein includes intravenous, intramuscular, subcutaneous, rectal, vaginal, or intraperitoneal administration. Peripheral systemic delivery by intravenous or intraperitoneal or subcutaneous injection is preferred. Suitable vehicles for such injections are straightforward in the art.

The pharmaceutical composition typically must be sterile and stable under the conditions of manufacture and storage in the container provided, including, e.g., a sealed vial or syringe. Therefore, pharmaceutical compositions can be sterile-filtered after making the formulation, or otherwise made microbiologically acceptable. A typical composition for intravenous infusion could have a volume as much as 250-1000 ml of fluid, such as sterile Ringer's solution, physiological saline, dextrose solution, and Hank's solution, and a therapeutically effective dose, (e.g., 1 to 100 mg/mL, or more) of antibody concentration. The dose can vary depending on the type and severity of the disease. As is well known in the medical arts, dosages for any one subject depend upon many factors, including the patient's size, body surface area, age, the particular compound to be administered, sex, time and route of administration, general health, and other drugs being administered concurrently. A typical dose can be, for example, in the range of from about 0.001 to about 1000 μg; however, doses below or above this exemplary range are envisioned, especially considering the aforementioned factors. The daily parenteral dosage regimen can be about 0.1 μg/kg to about 100 mg/kg of total body weight, preferably from about 0.3 μg/kg to about 10 mg/kg, more preferably from about 1 μg/kg to 1 mg/kg, and even more preferably, from about 0.5 to 10 mg/kg body weight per day. Progress can be monitored by periodic assessment. For repeated administrations over several days or longer, depending on the condition, the treatment is repeated until a desired suppression of disease symptoms occurs. However, other dosage regimens can be useful, and are not excluded herefrom. The desired dosage can be delivered by a single bolus administration, by multiple bolus administrations, or by continuous infusion administration of antibody, depending on the pattern of pharmacokinetic decay that the practitioner wishes to achieve.

These suggested amounts of antibody are subject to a great deal of therapeutic discretion. The key factor in selecting an appropriate dose and scheduling is the result obtained. Factors for consideration in this context include the particular disorder being treated, the particular mammal being treated, the clinical condition of the individual patient, the cause of the disorder, the site of delivery of the antibody, the particular type of antibody, the method of administration, the scheduling of administration, and other factors known to medical practitioners.

Therapeutic agents of the invention can be frozen or lyophilized for storage and reconstituted in a suitable sterile carrier prior to use. Lyophilization and reconstitution can lead to varying degrees of antibody activity loss. Dosages can have to be adjusted to compensate. Generally, pH between 6 and 8 is preferred.

Desacyl ghrelin plays a role in neuroendocrine, metabolic, and other related disorders or diseases (Broglio et al., Journal of Clinical Endocrinology & Metabolism 89(6):3062-3065, 2004; Gauna et al., Journal of Clinical Endocrinology & Metabolism 89(10):5035-5042, 2004; Asakawa et al., Gut 54(1):18-24, 2005; Chen et al., Gastroenterology 129(1):8-25, 2005). Therefore, a pharmaceutical composition comprising an anti-desacyl ghrelin monoclonal antibody of the invention can be used to treat such disorders or can be useful for the treatment of conditions wherein the presence of desacyl ghrelin causes or contributes to undesirable pathological effects, or decrease of desacyl ghrelin levels has a therapeutic benefit in mammals. If desired, an anti-desacyl ghrelin antibody could also be engineered to increase the half-life of this peptide, thus potentially prolonging its time of action.

The use of an anti-desacyl ghrelin monoclonal antibody of the present invention, or combinations thereof, for treating or preventing at least one of the aforementioned disorders in which desacyl ghrelin activity is detrimental or which benefits from decreased levels of bioactive desacyl ghrelin, is contemplated herein. Additionally, the use of an anti-desacyl ghrelin monoclonal antibody of the present invention for use in the manufacture of a medicament for the treatment of at least one of the aforementioned disorders is contemplated.

As used herein, the terms “treatment”, “treating”, and the like, refer to obtaining a desired pharmacologic and/or physiologic effect. The effect can be prophylactic in terms of completely or partially preventing a disease or symptom thereof, and/or can be therapeutic in terms of a partial or complete cure for a disease and/or adverse affect attributable to the disease. The term “treatment” as used herein includes administration of a compound of the present invention for treatment of a disease or condition in a mammal, particularly in a human, and includes: (a) preventing the disease from occurring in a subject predisposed to the disease but has not yet been diagnosed as having it; (b) inhibiting the disease, i.e., arresting its development; and (c) relieving the disease, i.e., causing regression of the disease or disorder or alleviating symptoms or complications thereof. Dosage regimens can be adjusted to provide the optimum desired response (e.g., a therapeutic or prophylactic response). For example, a single bolus can be administered, several divided doses can be administered over time, or the dose can be proportionally reduced or increased as indicated by the exigencies of the therapeutic situation.

The following examples are offered for illustrative purposes only, and are not intended to limit the scope of the present invention in any way.

### EXAMPLE 1

### Anti-Desacyl Ghrelin Fab and Monoclonal Antibody Synthesis

### Fab Synthesis

The CDR and framework sequences disclosed herein are identified from clones of Fab fragments isolated from antibody libraries generated from antibody RNA created by immunized C57B16 wild-type mice using Omniclonal™ antibody technology (Biosite®, San Diego, Calif.). The mice are immunized with human ghrelin acylated with n-octanoic acid at the His residue at position 9 (SEQ ID NO: 17). To improve the immunogenicity of this peptide, keyhole limpet hemocyanin is conjugated to the peptide through a C-terminal cysteine according to standard methods.

Table 1 shows the nucleic acid and corresponding amino acid sequences of the LCVR and CDRs 1, 2, and 3, contained therein, as well as the HCVR and CDRs 1, 2, and 3 contained therein, of Fab 5611. The CDR nucleic acid coding regions within the LCVR and HCVR are underlined.

TABLE 1

Fab 5611 Nucleic Acid and Amino Acid Sequences

| SEQ | | |
|---|---|---|
| NO: | DESCRIPTION | SEQUENCE |
| 1 | DNA LCVR | TCTACTGCAGCTTGGGCAGACCTTGTGCTGACACAGTCTCCTGCTTCCTTAGCTGTAT |
| | | CTCTGGGGCAGAGGGCCACCATCTCATGCAGGGCCAGCAAAAGTGTCAGTACATCTGG |
| | | CTATAGTTATATGCACTGGTACCAACAGAAACCAGGACAGCCACCCAAACTCCTCATC |
| | | TATCTTGCATCCAACCTAGAATCTGGGGTCCCTGCCAGGTTCAGTGGCAGTGGGTCTG |
| | | GGACAGACTTCACCCTCAACATCCATCGTGTGGAGGAGGAGGATGCTGCAACCTATTA |
| | | CTGTCAGCACAGTAGGGAGCTTCCGTACACGTTCGGAGGGGGGACCAAGCTGGAAATA |
| | | AAACGGGCTGATGCTGCACCAACTGTATCCATCTTCCCACCATCCAGTGACCAGTTAA |
| | | CATCTGGAGGTGCCTCAGTCGTGTGCTTCTTGAACAACTTCTACCCCAAAGACATCAA |
| | | TGTCAAGTGGAAGATTGATGGCAGTGAACGACAAAATGGCGTCCTGAACAGTTGGACT |
| | | GATCAGGACAGCAAAGACAGCACCTACAGCATGAGCAGCACCCTCACGTTGACCAAGG |
| | | ACGAGTATGAACGACATAACAGCTATACCTGTGAGGCCACTCACAAGACATCAACTTC |
| | | ACCCATTGTCAAGAGCTTCAACAGGAATGAG |
| 2 | AA LCVR | STPAWADLVLTQSPASLAVSLGQRATISCRASKSVSTSGYSYMHWYQQKPGQPPKLLI |
| | | YLASNLESGVPARFSGSGSGTDFTLNIHPVEEEDAATYYCQHSRELPYTFGGGTKLEI |
| | | KRADAAPTVSIFPPSSEQLTSGGASVVCFLNNFYPKDINVKWKIDGSERQNGVLNSWT |
| | | DQDSKDSTYSMSSTLTLTKDEYERHNSYTCEATHKTSTSPIVKSFNRNE |
| 3 | DNA LC CDR1 | AGGGCCAGCAAAAGTGTCAGTACATCTGGCTATAGTTATATGCAC |
| 4 | AA LC CDR1 | RASKSVSTSGYSYMH |
| 5 | DNA LC CDR2 | CTTGCATCCAACCTAGAATCT |
| 6 | AA LC CDR2 | LASNLES |
| 7 | DNA LC CDR3 | CAGCACAGTAGGGAGCTTCCGTACACG |
| 8 | AA LC CDR3 | QHSRELPYT |
| 9 | DNA HCVR | CAGGTTCAGCTGCAACAGTCTGAGGCTGAGCTGGTGAGGCCTGGGTCCTCAGTGAAGA |
| | | TTTCCTGCAAGGCTTCTGGCTATGCATTCAGTAACTACTGGATGAACTGGGTGAAGCA |
| | | GAGGCCTGGACAGGGTCTTGAGTGGATTGGACAGATTTATCCTGGAGATGGTGATACT |
| | | AAATACAATGGAAAGTTCAAGGGTAAAGCCACATTGACTGCAGACAAATCCTCCAGCT |
| | | CAGCCTACATGCAGCTCAGCAGCCTAACATCTGAGGACTCTGCGGTCTATTTCTGTGT |
| | | GATTACTACGGTAGTAGGAGGGGACTCCGATGTCTGGGGCGCAGGGACCACGGTCACC |
| | | GTCTCCTCAGCCAAAACGACACCCCCA |
| 10 | AA HCVR | QVQLQQSEAELVRPGSSVKISCKASGYAFSNYWMNWVKQRPGQGLEWIGQIYPGDGDT |
| | | KYNGKFKGKATLTADKSSSSAYMQLSSLTSEDSAVYFCVITTVVGGDSDVWGAGTTVT |
| | | VSSAKTTPP |
| 11 | DNA HC CDR1 | GGCTATGCATTCAGTAACTACTGGATGAAC |
| 12 | AA HC CDR1 | GYAFSNYWMN |
| 13 | DNA HC CDR2 | CAGATTTATCCTGGAGATGGTGATACTAAATACAATGGAAAGTTCAAGGGT |
| 14 | AA HC CDR2 | QIYPGDGDTKYNGKFKG |
| 15 | DNA HC CDR3 | ACTACGGTAGTAGGAGGGGACTCCGATGTC |
| 16 | AA HC CDR3 | TTVVGGDSDV |
| 17 | Human Ghrelin | GSSFLSPEHQRVQQRKESKKPPAKLQPX |
| | AA | |

### Monoclonal Antibody Synthesis

Cloning of E8 mouse IgG1 monoclonal antibody is performed as follows.

Fab 5611 is used as a template to PCR amplify the heavy and light chain variable domains from the Fab. The following primers are designed and synthesized:

| | |
|---|---|
| 5611HCF (heavy chain forward primer) | |
| tccaggatccaccggtcaggttcagctgcaacagtctgag | (SEQ ID NO: 18) |
| 5611 HCR (heavy chain reverse primer) | |
| ccaggggctagcggatagacagatgggggtgtcgt | (SEQ ID NO: 19) |
| 5611 LCF (light chain forward primer) | |
| tccaggatccaccggtgaccttgtgctgacacagtctcct | (SEQ ID NO: 20) |
| 5611LC (light chain reverse primer) | |
| gcagaattcggtttaaactcactaacactcattcctgttgaagctcttgac | (SEQ ID NO: 21) |

The resulting PCR-amplified fragment for the heavy chain variable domain is digested with BarnHI and NheI and cloned into a BamHI/NheI cut expression vector containing the Kappa signal for secretion and the constant domain of mouse IgG1. The resulting PCR-amplified fragment for the light chain variable domain is cut with BamHI and EcoRI and cloned into a BamHI/EcoRI cut expression vector containing the Kappa signal for secretion. The full-length heavy and light chain constructs are sequence confirmed, and used for production of the E8 mouse IgG1 mAb. All procedures employ standard molecular biological cloning/expression techniques.

### EXAMPLE 2

### ELISA Assay

Desacyl ghrelin is dried onto the surface of a Greiner MultiBind microtiter plate

(450-655061) by adding 60 uL of a 0.4 μg/ml (in H₂O) solution to each well. The assay plate is placed in a dry 37° C. incubator overnight. The next day, the assay plate is washed (wash buffer: 0.1% Tween 20, Tris-buffered saline (TBS)), and blocked with casein/PBS (Pierce 37528).

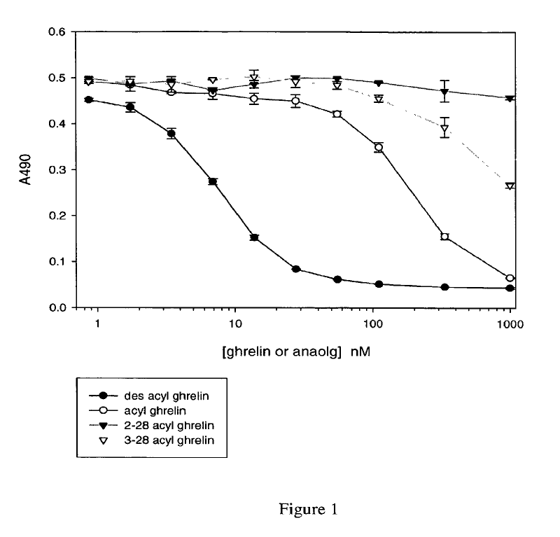

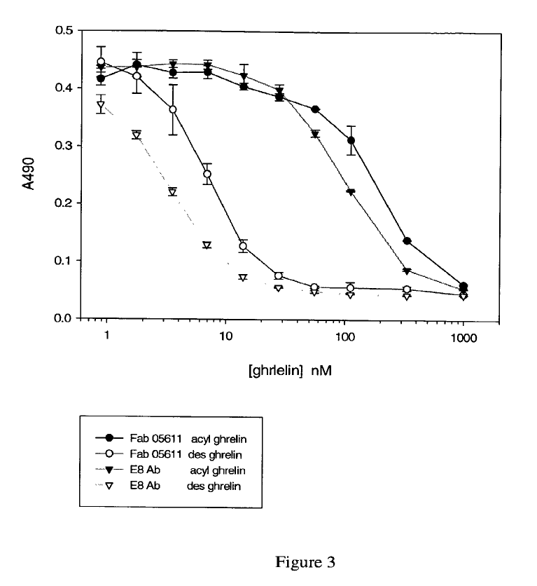

Ghrelin or ghrelin analogs are combined at various concentrations (see FIGS. 1-3) with Fab 5611 at 10 nM, or with the E8 monoclonal antibody at 10 nM (see FIG. 3), in casein/PBS. “2-28 acyl ghrelin” and “3-28 acyl ghrelin” in FIG. 1 refer to acyl ghrelins (SEQ ID NO: 17) missing the first one or two amino acids at the N-terminal end of the molecule, respectively. In FIG. 2, “1-8 (cys) desacyl” refers to a desacyl ghrelin fragment consisting of amino acids 1-8 of SEQ ID NO: 17, with an additional cysteine residue at the C-terminus. “4-28 (cys)” refers to a ghrelin fragment consisting of amino acids 4-28, also with an additional cysteine residue at the C-terminus. “9-28” refers to a ghrelin fragment consisting of amino acids 9-28 of SEQ ID NO: 17. Dilutions of the ghrelin, ghrelin analogs, and Fab are made in casein/PBS. These mixtures are incubated in a separate plate for 1 hour at room temperature.

The blocking solution is removed from the assay plate and 50 uL of the ghrelin/Fab mixture are added to the assay plate in duplicate. This is allowed to sit for 30 minutes at room temperature. The assay plate is washed 3 times and then goat, anti-mouse kappa-HRP (Southern Biotechnology 1050-05 at a 1:2000 dilution) is added. This is incubated for 1 hour at room temperature. The plate is washed 4 times and developed with OPD substrate (Sigma P-6912). The reaction is stopped with 100 uL of 1 N HCl, and the absorbance of the wells is read at 490 nm (Molecular Device SpectraMax250).

If Fab 5611 binds to ghrelin or a ghrelin analog prior to the mixture being added to the assay plate, then there will be less Fab 5611 available to bind to the desacyl ghrelin coated on the plate. This results in a reduction in the absorbance at 490 nm after incubation with goat, anti-mouse kappa-HRP antibody and reaction with OPD substrate.

As shown in FIG. 1, more of the acyl ghrelin than of the desacyl ghrelin is required to reduce the absorbance signal for Fab 5611. The data also show that if the first (N-terminal) amino acid of acyl ghrelin is removed (2-28 acyl ghrelin), it binds to Fab 5611 even more poorly.

The data in FIG. 2 show that both the 1-8 (cys) desacyl ghrelin and 1-28 desacyl ghrelin bind to Fab 5611, and that more 1-8 (cys) desacyl ghrelin is required to reduce the absorbance signal compared with 1-28 desacyl ghrelin. The data also show that the 4-28 (cys) and 9-28 fragments do not bind to Fab 5611 at the concentrations tested, indicating that Fab 5611 binds preferentially with desacyl ghrelin, and that the N-terminal amino acid(s) is(are) very important to the binding.

If the E8 antibody binds to ghrelin or a ghrelin analog prior to adding the mixture to the assay plate, then there will be less E8 antibody available to bind to the desacyl ghrelin coated on the plate. This results in a reduction in the absorbance at 490 nm after incubation with goat, anti-mouse kappa-HRP antibody and reaction with OPD substrate.

As shown in FIG. 3, both Fab 5611 and the E8 antibody recognize desacyl ghrelin significantly more than ghrelin.

Taken together, the data in FIGS. 1 and 2 demonstrate that Fab 5611 binds to an epitope residing within amino acids 1-8 of desacyl ghrelin. The data in FIG. 3 show that both Fab 5611 and the E8 antibody preferentially bind to desacyl ghrelin compared to acylated ghrelin.

### EXAMPLE 3

### Affinity Measurement of Monoclonal Fabs and Antibodies

The affinity (K_{D}) and Kₒₙ and K_{off} rates of anti-desacyl ghrelin Fabs and monoclonal antibodies of the present invention are measured using a BLAcore® 2000 instrument containing a CM5 sensor chip. The BIAcore® utilizes the optical properties of surface plasmon resonance to detect alterations in protein concentration of interacting molecules within a dextran biosensor matrix. Except where noted, all reagents and materials are purchased from BIAcore® AB (Upsala, Sweden). All measurements are performed at 25° C. Samples containing rat or human desacyl ghrelin are dissolved in HBS-EP buffer (150 mM sodium chloride, 3 mM EDTA, 0.005% (w/v) surfactant P-20, and 10 mM HEPES, pH 7.4). A capture antibody, goat anti-mouse Kappa (Southern Biotechnology, Inc), is immobilized onto flow cells using amine-coupling chemistry. Flow cells (1-4) are activated for 7 minutes with a 1:1 mixture of 0.1 M N-hydroxysuccinimide and 0.1 M 3-(N,N-dimethylamino)propyl-N-ethylcarbodiimide at a flow rate of 10 μl/min. Goat anti-mouse Kappa (30 μg/mL in 10 mM sodium acetate, pH 4.5) is manually injected over all 4 flow cells at a flow rate of 10 μL/min. The surface density is monitored and additional goat anti-mouse Kappa is injected if needed to individual cells until all flow cells reach a surface density of 4500-5000 response units (RU). Surfaces are blocked with a 7 minute injection of 1 M ethanolamine-HCl, pH 8.5 (10 μL/min). To ensure complete removal of any noncovalently bound goat anti-mouse Kappa, 15 μL of 10 mM glycine, pH 1.5, are injected twice. Running buffer used for kinetic experiments contains 10 mM HEPES, pH 7.4, 150 mM NaCl, 0.005% P20.

Collection of kinetic binding data is performed at maximum flow rate (100 μL/min) and a low surface density to minimize mass transport effects. Each analysis cycle consists of: (i) capture of 300-350 RU of Fabs (BioSite) by injection of 5-10 μL of 5 μg/ml solution over flow cells 2, 3, and 4 for different Fabs at a flow rate of 10 μL/min., (ii) 200 μL injection (2 min) of human desacyl ghrelin (concentration range of 50 nM to 1.56 nM in 2-fold dilution increments) over all 4 flow cells with flow cell 1 as the reference flow cell, (iii) 10 min dissociation (buffer flow), (iv) regeneration of goat anti-mouse Kappa surface with a 15 sec injection of 10 mM glycine, pH 1.5, (v) a 30 sec blank injection of running buffer, and (vi) a 2 min stabilization time before start of next cycle. Signal is monitored as flow cell 2 minus flow cell 1, flow cell 3 minus flow cell 1 and flow cell 4 minus flow cell 1. Samples and a buffer blank are injected in duplicate in a random order. Data are processed using BIAevaluation 3.1 software and data are fit to a 1:1 binding model in CLAMP global analysis software.

K_{ON}, K_{OFF}, and K_{D} for Fab 5611 with desacyl human ghrelin are shown in Table 2.

TABLE 2
Fab 5611/Des-acyl Human Ghrelin

| Fab | K_{ON} | K_{OFF} | K_{D} (K_{OFF}/K_{ON}) (M) |
|---|---|---|---|
| 5611 | 8.77 × 10⁵ | 1.21 × 10⁻⁴ | 1.38 × 10⁻¹⁰ |

K_{ON}, K_{OFF}, and K_{D} for the E8 monoclonal antibody with desacyl human ghrelin are shown in Table 3.

TABLE 3
E8/Des-acyl Human Ghrelin

| Antibody | K_{ON} | K_{OFF} | K_{D} (K_{OFF}/K_{ON}) (M) |
|---|---|---|---|
| E8 | 9.28 × 10⁵ | 2.10 × 10⁻⁴ | 2.26 × 10⁻¹⁰ |

### EXAMPLE 4

### FLIPR In Vitro Activity Assay

The in vitro FLIPR® Calcium Assay system (Molecular Devices) is used with cells stably transfected to express a human desacyl ghrelin receptor. This assay evaluates changes in intracellular calcium as a means of detecting desacyl ghrelin/receptor binding and signaling in the presence or absence of a Fab of the invention. This functional assay can also be used to further map the location of the epitope to which the monoclonal antibodies or antigen-binding portions thereof of the invention bind.

Cells are grown in growth medium ((DMEM/F12 (3:1), 5% fetal bovine serum, with selection agent) to about 50-90×10⁶ cells per T-150 flask. The cells are then trypsinized, washed, and distributed into Biocoat black poly-D-lysine coated plates (60,000 cells in 100 μl growth medium per well). The cells are incubated for about 20 hours at 37° C. in 5% CO₂. The medium is removed from the plate and 150 μl HBSS (Gibco 14025) are added to each well and then removed. Dye is then loaded into the cells by adding to each well 50 μl loading buffer (5 μM Fluo-4AM (Molecular Devices), 0.05% Pluronic in FLIPR buffer (Hank's Balanced Salt with calcium (HBSS, Gibco 14025) and 0.75% BSA (Gibco)). The plate is further incubated at 37° C. in 5% CO₂ for one hour. The wells are then washed twice with HBSS, and 50 μl FLIPR buffer are then added per well.

Samples are prepared by combining 7.2 μl calcium concentrate (CaCl₂-2H₂O in water at 3.7 mg/ml mixed 1:1 with HB SS and filter sterilized) with 60 μl Fab (of varying concentrations), and 16.8 μl desacyl ghrelin in 3.75% BSA/50% HBSS. The final concentration of the sample solution is 0.75% BSA, and calcium at approximately the same concentration as in the FLIPR buffer. The cell plate is shaken for 15 seconds prior to loading it into the FLIPR instrument. Fifty microliters of the sample solution are added to the 50 μL FLIPR buffer in the well with the cells and read by a Fluorometric Imaging Plate Reader (Molecular Devices).

If there is no Fab, or an irrelevant antibody, present in the solution, the full-length desacyl ghrelin will be free to bind the receptor on the cells and signal transduction will occur, resulting in comparatively high values in the assay. If a Fab is present that binds to the full-length desacyl ghrelin in the solution, then the binding of the full-length desacyl ghrelin to the receptor is inhibited and signal transduction is thereby inhibited, resulting in comparatively lower values in the assay.

To use the assay to determine the Fab epitope, an active desacyl ghrelin fragment can be substituted for the full-length desacyl ghrelin. If a Fab is present that binds to the desacyl ghrelin fragment in the solution, then the binding of the desacyl ghrelin fragment to the receptor is inhibited and signal transduction is thereby inhibited, resulting in comparatively lower values in the assay.

Additionally, the Fab epitope can be determined by combining an inactive desacyl ghrelin fragment with the Fab to see if it will block the ability of the Fab to inhibit binding of the full-length desacyl ghrelin. If a peptide (i.e., a fragment of desacyl ghrelin) is added to the solution and the Fab binds the peptide, then the full-length desacyl ghrelin is not prevented from binding the receptor, signal transduction is not inhibited, and the values in the assay are comparatively high.

The invention being thus described, it is obvious that the same can be varied in many ways. Such variations are not to be regarded as a departure from the spirit and scope of the present invention, and all such modifications as would be obvious to one skilled in the art are intended to be included within the scope of the following claims.

1-22. (canceled)

23. A monoclonal antibody or antigen-binding fragment thereof, comprising a light chain variable region having the amino acid sequence shown in SEQ ID NO: 2 and a heavy chain variable region having the amino acid sequence shown in SEQ ID NO: 10.

24. The monoclonal antibody or antigen-binding fragment thereof of claim 23, comprising a heavy chain constant region selected from the group consisting of IgG1, IgG2, IgG3, IgG4, IgA, IgE, IgM, and IgD.

25. The monoclonal antibody or antigen-binding fragment thereof of claim 23, comprising a kappa or lambda light chain constant region.

26. The monoclonal antibody or antigen-binding fragment thereof of 23, wherein said antigen-binding fragment thereof is selected from the group consisting of a Fab fragment, a Fab′ fragment, a F(ab′)2 fragment, and a single chain Fv fragment.

27. A pharmaceutical composition, comprising said antibody or antigen-binding fragment thereof of claim 23, and a pharmaceutically acceptable carrier, diluent, or excipient.

28. A method of treating obesity, non-insulin dependent diabetes mellitus, Prader-Willi syndrome, hyperphagia, or impaired satiety in a human in need thereof, comprising administering to said human an effective amount of said monoclonal antibody of claim 23.


SEQUENCE LISTING (21 sequences)

SEQ ID NO: 1 - DNA, 669 bp, Mus sp.
tctactccagcttgggcagaccttgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgag

SEQ ID NO: 2 - protein, 223 aa, Mus sp.
STPAWADLVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNE

SEQ ID NO: 3 - DNA, 45 bp, Mus sp.
agggccagcaaaagtgtcagtacatctggctatagttatatgcac

SEQ ID NO: 4 - protein, 15 aa, Mus sp.
RASKSVSTSGYSYMH

SEQ ID NO: 5 - DNA, 21 bp, Mus sp.
cttgcatccaacctagaatct

SEQ ID NO: 6 - protein, 7 aa, Mus sp.
LASNLES

SEQ ID NO: 7 - DNA, 27 bp, Mus sp.
cagcacagtagggagcttccgtacacg

SEQ ID NO: 8 - protein, 9 aa, Mus sp.
QHSRELPYT

SEQ ID NO: 9 - DNA, 375 bp, Mus sp.
caggttcagctgcaacagtctgaggctgagctggtgaggcctgggtcctcagtgaagatttcctgcaaggcttctggctatgcattcagtaactactggatgaactgggtgaagcagaggcctggacagggtcttgagtggattggacagatttatcctggagatggtgatactaaatacaatggaaagttcaagggtaaagccacattgactgcagacaaatcctccagctcagcctacatgcagctcagcagcctaacatctgaggactctgcggtctatttctgtgtgattactacggtagtaggaggggactccgatgtctggggcgcagggaccacggtcaccgtctcctcagccaaaacgacaccccca

SEQ ID NO: 10 - protein, 125 aa, Mus sp.
QVQLQQSEAELVRPGSSVKISCKASGYAFSNYWMNWVKQRPGQGLEWIGQIYPGDGDTKYNGKFKGKATLTADKSSSSAYMQLSSLTSEDSAVYFCVITTVVGGDSDVWGAGTTVTVSSAKTTPP

SEQ ID NO: 11 - DNA, 30 bp, Mus sp.
ggctatgcattcagtaactactggatgaac

SEQ ID NO: 12 - protein, 10 aa, Mus sp.
GYAFSNYWMN

SEQ ID NO: 13 - DNA, 51 bp, Mus sp.
cagatttatcctggagatggtgatactaaatacaatggaaagttcaagggt

SEQ ID NO: 14 - protein, 17 aa, Mus sp.
QIYPGDGDTKYNGKFKG

SEQ ID NO: 15 - DNA, 30 bp, Mus sp.
actacggtagtaggaggggactccgatgtc

SEQ ID NO: 16 - protein, 10 aa, Mus sp.
TTVVGGDSDV

SEQ ID NO: 17 - protein, 28 aa, Homo sapiens
  MISC_FEATURE (28)..(28): Xaa at position 28 is Arg or is absent.
GSSFLSPEHQRVQQRKESKKPPAKLQPX

SEQ ID NO: 18 - DNA, 40 bp, Artificial
  heavy chain forward primer
tccaggatccaccggtcaggttcagctgcaacagtctgag

SEQ ID NO: 19 - DNA, 35 bp, Artificial
  heavy chain reverse primer
ccaggggctagcggatagacagatgggggtgtcgt

SEQ ID NO: 20 - DNA, 40 bp, Artificial
  light chain forward primer
tccaggatccaccggtgaccttgtgctgacacagtctcct

SEQ ID NO: 21 - DNA, 51 bp, Artificial
  light chain reverse primer
gcagaattcggtttaaactcactaacactcattcctgttgaagctcttgac